(19) United States
(12) Patent Application Publication
(10) Pub. No.: US 2018/0318750 A1
(43) Pub. Date: Nov. 8, 2018

(54) Method for Gas Separation, Purification and Clarification by FTrPSA

(71) Applicant: SICHUAN TECHAIRS CO., LTD., Chengdu, Sichuan (CN)

(72) Inventors: Yuming Zhong, Chengdu, Sichuan (CN); Kaili Liu, Chengdu, Sichuan (CN); Yun Chen, Chengdu, Sichuan (CN); Yueming Cai, Chengdu, Sichuan (CN)

(21) Appl. No.: 15/774,587

(22) PCT Filed: Feb. 17, 2017
(86) PCT No.: PCT/CN2017/073903

§ 371 Date: May 9, 2018

(30) Foreign Application Priority Data
Mar. 31, 2016 (CN) 201610196432.3

(51) Int. Cl. B01D 53/047; C10K 1/00; C10K 1/18; C10K 1/20; C07C 7/12; C07C 7/11; C07C 7/00; C10L 3/10; C01B 3/56; C01B 3/52; C01B 13/02

(52) U.S. Cl. CPC B01D 53/047; B01D 53/52; C10K 1/002; C10K 1/18; C10K 1/20; C07C 7/12; C07C 7/11; C07C 7/005; C10L 3/101; C10L 3/103; C01B 3/56; C01B 3/52; C01B 13/0259; B01D 2257/708; B01D 2257/7027; B01D 2256/24; B01D 2257/7022; B01D 2256/16; B01D 2257/304; B01D 2256/12; B01D 2257/102; B01D 2256/245; C10K 1/004

(57) ABSTRACT

A method for gas separation, purification and clarification by FTrPSA uses the temperature and pressure of different raw gases as well as the differences in adsorption separation coefficients and physicochemical properties among all components in the raw gases at a temperature range of −80-200° C. and a pressure range of 0.03-4.0 Mpa, regulates the adsorption or desorption regeneration operation in the PSA cycle process by coupling various separation methods, and expands the adsorption theory that the PSA or TSA separation process is limited to the cyclic operation of adsorption and desorption regeneration through pressure or temperature changes, thus realizing the gradient utilization of energy in the process of gas separation, purification and clarification as well as the easy-to-match and easy-to-balance cyclic operation of adsorption and desorption regeneration in the process of intercooling & shallow-cooling and medium & high-temperature PSA separation to separate, purify and clarify various raw gases.

### CROSS REFERENCE OF RELATED APPLICATION

This is a U.S. National Stage under 35 U.S.C 371 of the International Application PCT/CN2017/073903, filed Feb. 17, 2017, which claims priority under 35 U.S.C. 119(a-d) to CN 201610196432.3, filed Mar. 31, 2016.

### BACKGROUND OF THE PRESENT INVENTION

### Field of Invention

The present invention belongs to the field of gas separation, purification and clarification, and more specifically relates to a method for gas separation, purification and clarification by FTrPSA.

### Description of Related Arts

Gas separation is the separation of a gas mixture containing various components to obtain one or several components as a product gas or removal of one or several components as a clarified gas (or acceptable effluent gas), for example, separation and extraction of ethylene (C2H4) propylene (C3H6) from ethylene cracking gas, clarification of benzene naphthalene removed from coke oven gas, recycling of component C2 and above (C2 and above) of ethylene-ethane and the like from ethylene and refinery tail gas, extraction of product hydrogen (H2) from hydrogen containing refinery dry gas, extraction of oxygen (O2) nitrogen (N2) from air, removal of hydrogen sulfide (H2S) from hydrogen-rich gas source, removal of volatile organic compounds (VOC) from biopharmaceutical tail gas, desulfurization, decarbonization and denitrification from flue gas and the like. Gas separation has been widely applied in petrochemical industry, coal chemical industry, biological medicine, industry of fine chemicals, electronics, electric power generation, coal, food, articles of everyday use and other industries, especially in atmospheric environment protection, comprehensive treatment of industrial waste gas and energy saving and emission reduction in industrial production where gas separation has become the most important technical method and means.

Gas separation generally requires removal of some impurity components from a mixed raw gas containing various components, the content (concentration, all by volume %, similarly below) of these impurity components is less than 1-10%, and this separation process is called clarification, i.e. removal; the rest of separation process is called separation and purification or extraction. The technical methods of gas separation comprise distillation, absorption, extraction, membrane separation, drying, adsorption and other unit operations of chemical engineering. Different separation methods are selected for treatment depending on the separated target components and mixed raw gas components and corresponding separation principles. Due to the complexity of treated raw gas components, concentration, temperature, pressure, treatment capacity and other conditions in most industrial practical applications, a single separation method is difficult to play a role, so various separation methods must be combined in most cases to be effectively applied to solve many technical bottlenecks in the process of gas separation, including expanding the application field, reducing energy consumption and cost, improving efficiency and product gas purity and the like.

Distillation is the most basic and mature unit operation of chemical engineering in the field of separation, the difference in the boiling points or volatilities of components in a mixture fluid (liquid and gas) is utilized to achieve the purpose of separation of the components from each other, comprising evaporation, rectification, cryogenic rectification, deep cooling (ultra-low temperature rectification) and other distillation processes. Although distillation has been widely used in the fields of petrochemical refining, industry of fine chemicals, coal chemical industry and the like, due to the existence of phase transition and the coexistence of the processes of heat exchange and mass transfer required in the form of repeated temperature rise and fall, its energy utilization ratio is relatively low, and the yield of the product cannot be improved while the purity of the product is improved. The distillation process is mainly treatment of liquid separation, but due to similar volatilities of organic solvents such as alcohols, ethers and esters, and components such as alkane and olefine or the separation and purification conditions such as formation of azeotrope, neither azeotropic rectification nor molecular rectification can effectively realize separation and purification; in a lot of fields of gas separation, purification and clarification such as removal and control of pollutants with dilute concentrations in atmosphere, comprehensive utilization of industrial tail gas and industrial gas preparation, distillation can be neither used as the most basic separation unit, nor used as the most common ultimate separation unit. In the fields of separation and clarification such as air separation, separation of ethylene cracking gas and separation of synthesis gas, widely used (ultra) low temperature rectification (deep cooling) is at the expense of high energy consumption, no effective utilization of self-contained energy of raw gas, and inexistence of effective alternative technology at present. Example 1, the application of rectification separation in petrochemical ethylene production: in the process of ethylene production by steam cracking with naphtha as a raw material, the ethylene cracking gas produced is a typical case of mixed gas recycled by rectification separation, extraction and clarification. The temperature of the cracking gas produced from an ethylene cracking furnace is up to 700-900° C., wherein the average ethylene content is 15-40%, propylene 8-16%, butadiene 2-5%, aromatics (benzene, toluene, xylene) 2-5%, methane 5-15% and hydrogen 1-5%, and other components are nitrogen (N2), carbon dioxide (CO2), carbon monoxide (CO), ethane, propane, various other light hydrocarbons, light and heavy oil, sulfur and water. The boiling points of various components in ethylene cracking gas vary a lot, from low boiling point components such as H2, N2, CO, methane and the like with boiling points ranging from (−270 to −107° C.), to medium-and-high boiling point components such as ethylene, ethane, propylene, water, naphtha, heavy oil and the like with boiling points ranging from (70 to 300° C.). For industrial separation of ethylene cracking gas, after pretreatments such as quenching, pressurization, alkali washing and drying, the ethylene cracking gas successively passes a series of cryogenic and shallow cooling rectification units such as −110-−70° C. deep cooling and demethanizing tower (low temperature rectification) and −70-−50° C. deethanizing tower, followed by shallow cooling and rectification at normal temperature, so that an ethylene-propylene product is finally obtained through the difference in boiling points of all the easily liquefied components. That is to say, the temperature span from a high temperature raw gas, i.e. the ethylene cracking gas, at the temperature of 700-900° C. at the cracking furnace outlet into material low temperature rectification (deep cooling) is very large, the self-contained energy of the material gas is hardly fully and effectively utilized, and the energy required from the outside is very large. In addition, in order to further improve the efficiency of low temperature rectification in each work section, the reflux ratio needs to be constantly increased, so that light and heavy components in the rectification tower are returned to their separation zones effective for separation through various condensers, coolers and reboilers, thus improving the separation efficiency. Those components with low boiling points, such as H2, N2, CO, methane and the like which are not easily liquefied at low temperature or shallow cooling or normal temperature, form non-condensable gases, carrying small amounts of medium-and-high boiling point components such as C2 and above throughout the main deep cooling and demethanizing tower procedures, resulting in meaningless increase in the energy consumption of these two procedures. Small amounts of the non-condensable gases still continue to “play a bit role” in the subsequent low temperature or shallow cooling or normal temperature rectification procedure, resulting in energy waste in each rectification work section, increase in the reflux ratio, and increase in the operating cost. These non-condensable gases eventually form ethylene tail gas (dry gas) generally used as fuel gas directly, but effective components such as a large amount of H2 cannot be returned through recycling to a refinery hydrogenation section requiring a large amount of hydrogen. If these non-condensable gas components need to be recycled, further deep cooling (ultra-low temperature rectification) separation at lower temperature (less than −107° C.) needs to be further used for recycling. It is obvious that the rectification separation process of ethylene cracking gas is actually a process in which the temperature continually and repeatedly rises and falls, the effective utilization of energy has become the key to rectification separation of the ethylene cracking gas, and it is also a work section with the maximum cost of ethylene-propylene production in the total cost, up to more than 70%. The technological transformation and innovation of the procedure of ethylene cracking gas separation are mostly concentrated on the effective utilization of energy. However, only a series of rectification procedures themselves are improved, the huge energy carried by ethylene cracking gas itself cannot be effectively utilized, due to the limitation of the rectification separation principle itself, these improvements and innovations still cannot change the current situation in the existing procedure of ethylene cracking and gas separation that the operation energy consumption and production cost remain at a high level. At present, for the formed ethylene tail gas, the separation methods of oil absorption and adsorption can be used to recycle a small amount of ethylene (components of C2 and above) and H2 in the ethylene tail gas instead of the conventional method of cryogenic separation and recycling of ethylene tail gas. Example 2, separation and clarification of coke oven gas in the field of coal chemical industry: Unlike separation of ethylene cracking gas in example 1, coke oven gas separation is a typical clarification process of removing a small amount of harmful impurities therein. The temperature of coke oven gas produced from the coking section is also up to 650-800° C., and its typical components are H2 (20-60%), methane (10-40%), CO (5-20%), a certain amount of tar vapor, benzene vapor, naphthalene vapor and the like. Coke oven gas can be used for iron and steel production, used by urban residents and used as the raw material for production of synthetic ammonia, methanol, hydrogen, natural gas and the like. Before the integrated utilization of coke oven gas, a lot of impurities comprising tar, naphthalene, hydrogen sulfide (H2S), benzene, hydrocyanic acid (HCN) and the like need to be removed from coke oven gas. Thus, coke oven gas can become the qualified fuel that can be finally used for civilian use only through the conventional crude removal of naphthalene tar by initial cooling and crystallization, absorption desulfurization and catalytic elution of ammonia, final cooling, benzene and naphthalene removal by rectification, desulphurization and naphthalene removal by deep adsorption, as well as a series of complex and numerous cooling energy and heat exchange systems and other separation steps. However, since there are various impurities to be removed from coke oven gas, some of the physical properties of these impurities such as boiling point, solubility, equilibrium adsorption capacity and the like are very close and some are very different, the contents of all impurity components are less than 10%, and the impurities will interfere with each other in terms of efficiency in their separation and clarification sections. Therefore, if one step in the process of separation and clarification of coke oven gas has not been handled properly, or the components in the raw gas fluctuate greatly, the efficiency of the next separation and clarification procedure will be greatly affected, so that the total cost of separation and clarification of coke oven gas is increased. In addition, through the separation and clarification of coke oven gas, no clean coke oven gas product can be obtained by directly using the ultra-low temperature or low temperature or shallow cooling rectification procedure just like separation of ethylene cracking gas. Because the coke oven gas contains a lot of low boiling point components, while in cryogenic separation, a small amount of impurity components such as sulfides, ammonia, water, naphthalene (crystallized) and the like which are easily liquified or crystallized and cured at low temperature do great harm such as corrosion or blockage to cryogenic equipment, causing a potential safety hazard in cryogenic operation. Therefore, the separation and clarification of coke oven gas is a typical case covering a variety of gas separation and clarification methods, and many complex and inefficient technical problems in the clarification separation of coke oven gas can be overcome only by improvements from the perspective of integration of all the work sections.

Absorption is a “like dissolves like” physical mass transfer process, and different solubilities of all components in a mixture fluid (gas) imported in a certain imported absorption solvent are utilized to achieve the purpose of separation of components from each other. The process of absorption and separation is relatively simple and widely applied in gas separation, purification and clarification such as desulphurization, denitration, decarbonization, removal of organics and impurities, recycling of the components such as refinery dry gas C2 and above. Due to the consumption of more absorption solvents, the consumption of energy and materials is higher, and it is difficult to meet the requirement of higher clarification degree of impurity removal or product gas purity. For the condition of low concentration of impurities to be removed from the raw gas, the absorption efficiency is low, and the important mission of ultimate separation procedure cannot be undertaken. Example 1, the oil absorption and recycling of the components such as refinery dry gas C2 and above: refinery dry gas is a generic term of the gases obtained during petroleum processing, which mainly comprises catalytic cracking dry gas, hydrocracking dry gas, coking dry gas, reforming dry gas, ethylene tail gas and the like, and the components, temperatures and pressures thereof are different from each other. Industrially, different dry gases are mixed together for treatment and application, such as recycling of C2 and above and H2 therein. The average components of refinery dry gas are H2 (10-40%), methane (5-20%), ethylene (1-20%), ethane (1-20%), C3 and above (1-15%), as well as N2, CO2, H2S, O2 and other impurities, and the temperature ranges from −70 to 80° C. In practical applications, intercooling oil absorption and shallow-cooling oil absorption can be carried out separately for recycling of C2 and above in refinery dry gas within −70-20° C. and 3-4 MPa according to the “like dissolves like” property between C2 and above and oil (C3-C6 light oil and C6+ medium oil). Since the concentration, pressure and temperature of C2 and above in refinery dry gas fluctuate greatly, the concentration for C2 and above is smaller, for example, less than 10%, the concentration and partial pressure of C2 and above in the raw gas are not high, not very different from the corresponding saturated vapor pressure of C2 and above, the driving force of absorption is also small, thus the efficiency of cold oil absorption is not high. To improve the efficiency, it is necessary to increase the absorption pressure or further reduce the absorption temperature so that the energy consumption is substantially increased. In addition, the absorption liquid which is rich in C2 and above and formed by cold oil absorption needs to regenerate an absorbent and emit a concentrated gas rich in C2 and above by depressurization or heating-up under normal working conditions, the absorbent is recycled, and the concentrated gas of C2 and above is supplied into the next step of procedure for refining. Therefore, the great pressure drop or temperature difference in the processes of absorption and regenerative cycle cannot be used effectively, and the energy consumption is higher; example 2, removal and clarification of H2S in a hydrogen-rich gas source: the hydrogen content in the hydrogen-rich gas source is generally more than 50%, comprising reformed gas of natural gas and water vapor, methanol cracking gas, reformed gas, catalytic dry gas, hydrorefining, hydrocracking and the like. These gas sources usually contain a certain amount of H2S, and the temperature is usually in the range of 50−300° C. H2S impurities have great negative effects on the use of hydrogen-rich gas source in subsequent procedures, for example, corrosion of equipment pipelines, excessive emission of sulfur dioxide (SO2) from fuel gas, catalyst and adsorbent poisoning and the like. The general method of H2S removal is absorption, comprising chemical absorption and physical absorption. Organic amines are used as absorbents for physical absorption in the industry, and the desulfurization rate is relatively high. However, first, there are often impurity components, such as C2 and above, carbon dioxide (CO2) and the like, which are easily absorbed in the hydrogen-rich gas source, there is the problem of competitive absorption with H2S absorption, leading to a decline in the desulphurization effect and a substantial increase in both the dosage of absorbent and the circulation and loss of regenerative load and absorbent, so that the absorption cost increases significantly; second, the temperature of the raw gas is higher, or the pressure is lower, but the absorption temperature is low, generally normal temperature, or the absorption pressure is higher, and as a result, the absorbed energy consumption is relatively high, and some energy carried by the raw gas itself cannot be fully utilized; third, since the H2S contents in most hydrogen-rich gas sources as raw gases are relatively low, the desulfurization efficiency by the absorption and separation method is very low, the reason is also the same as the recycling of refinery dry gas C2 and above in example 1, the absorbed H2S concentration and partial pressure are lower, as a result, the difference between its partial pressure and its saturated vapor pressure is too small, or there is even no difference, the necessary driving force for absorption is insufficient, the absorption efficiency is very low, or it is even impossible to absorb, fourth, H2S removal by the absorption and separation method is limited in depth which can only reach 10-100 ppm, and it is difficult to meet the requirement of less than 0.1-1 ppm.

Extraction is mainly applied to liquid separation, which means that different solubilities of all components in a mixed liquid in an imported extraction solvent are utilized to achieve the purpose of separation. It has the main advantages of simplicity and energy saving, and the method is widely applied to the separation and purification of rare metals, organic solvents and the like. Coupled with rectification, absorption and other separation technologies, some conditions such as rectification and absorption in azeotrope separation, low content of impurities to be removed and the like which are difficult to solve effectively can be solved. However, the requirement of higher clarification degree of removed impurities or higher purity of product components is still difficult to effectively meet. Compared with the field of gas separation, the application of extraction technology is seriously limited. Even if some gas separation conditions are usable, there are some problems similar to absorption, for example, excessive consumption and loss of extractant, no full utilization of self-contained energy of raw gas, not high extraction efficiency and the like.

Membrane separation is a recently fast developing separation technology which utilizes the difference in osmotic pressure or selectivity of all components in mixture fluid (gas or liquid) passing a certain membrane to achieve the purpose of separation. Compared with the conventional separation technologies such as rectification, absorption and extraction, membrane separation has the most important advantages of energy saving, simplicity and effectiveness, and membrane separation is widely applied in the fields of gas separation and purification, sea water desalination, sewage treatment, rare metal concentration and the like. Membrane separation has the greatest disadvantage that the membrane material is highly expensive and easily contaminated by impurities, resulting in its short service life and higher cost, and many pretreatment aids need to be set to protect the clean membrane itself from being contaminated. In addition, the manufacturing of the membrane material itself is underdeveloped in China, the quality cannot be guaranteed, thus greatly restricting the popularization and application of membrane separation technology. Currently, membrane separation cannot become a widely used basic unit operation for the time being.

Drying is a simple physical process which uses the difference in volatility or solubility of a (some) components in a mixed object (solid, gas or liquid) to achieve the purpose of separation by vaporizing or dissolving it by heat or cooling capacity. Drying is mainly removal of moisture and some volatile organic compounds (VOC) from solid. In the field of gas separation, drying is mainly to achieve the purpose of dewatering or VOC removal by the adsorption separation method.

The adsorption separation process is a chemical unit operation in which mixture fluid (gas or liquid) is treated by porous solid (adsorbent) to gather or condense one or more components contained in the mixture fluid on its surface to achieve the purpose of separation, The process is widely used in the fields of petrochemical industry, coal chemical industry, industry of fine chemicals, metallurgy, electronics, medicines, environmental protection, gas industry and the like, wherein liquid phase adsorption mainly treats liquid separation, and gas phase adsorption mainly treats the separation, purification and clarification of mixed gas. Gas adsorption mainly comprises two types, i.e. PSA and TSA, and there are also other types of adsorption, comprising temperature-pressure swing adsorption (TPSA) and adsorption coupled with other separation techniques. Gas adsorption separation is often considered as the ultimate unit operation which can extract high purity product gas and remove some impurities in depth, but cannot undertake basic separation tasks such as rectification, absorption and other conventional separation techniques. Also, the self-energy of the raw gas cannot be fully utilized either under many working conditions.

Pressure Swing Adsorption (PSA) means that a mixture gas is separated, purified and clarified at a certain temperature through the cyclic process of pressurized adsorption and depressurization (vacuum pumping) or desorption at normal pressure or flushing, replacement and regeneration. It can be seen that PSA achieves the purpose of adsorption and desorption by changing the pressure. Adsorption is often carried out in a pressure environment. PSA proposes a method of combining pressurization and depressurization, and the method is usually a cyclic operating system of adsorption-desorption (regeneration) consisting of pressurization-adsorption and depressurization-regeneration at a certain temperature. The volume of adsorbent adsorbed by the adsorbent increases as the pressure rises and decreases as the pressure drops. At the same time, the adsorbed gas is released to regenerate the adsorbent during the depressurization (drop to normal pressure or vacuum pumping), thus, the multi-component mixed gas is separated or clarified, the adsorbent can be regenerated without heat supply from outside, the pressure changes can be partially compensated by the method of multi-tower pressure sharing in the adsorption-regeneration cycle operation, and the loss of pressure drop is reduced. PSA has the advantages as follows: (1) the purity of the product is high; (2) PSA can generally work at room temperature and not very high pressure, the bed is regenerated without heating for energy saving and economy; (3) As a energy efficient and effective separation method, the equipment is simple and easy to operate and maintain, continuous cyclic operation is possible, and automation is fully achieved. (4) The cycle period is short, the adsorbent can be regenerated and repeatedly used, and the utilization rate is high. PSA is mainly used for gas separation, extraction and clarification under the complex working conditions for raw gas components with high gas consumption, such as recycling of H2/C2 and above from refinery drying gas, air separation, deep desulfurization and naphthalene removal of coke oven gas, preparation of high purity industrial gas, synthetic gas separation and the like. Despite some advantages as above, the conventional PSA has some significant disadvantages: (1) the product yield is low; (2) the contradiction between adsorption and desorption cannot be solved, the applications in many occasions such as desorption and clarification of trace impurities, adsorption and recycling of components with low partial pressure and the like are limited; (3) weakly polar impurities are difficult to adsorb, and highly polar trace impurities are difficult to desorb and regenerate; (4) as operating basically at normal temperature, the conventional PSA fails to effectively utilize the self-contained energy of the raw gases at a high or low temperature, and to achieve the cyclic operation of adsorption and desorption regeneration as well as the high efficiency of separation, purification and clarification; (5) there are many more sequencing valves, with a high failure rate.

Temperature Swing Adsorption (TSA) means the method of adsorption at a normal temperature or low temperature and desorption at a high temperature with the pressure kept constant. Obviously, for TSA, adsorption and desorption are carried out by changing the temperature. The TSA operation is carried out at the perpendicular line between a low temperature (usually at normal temperature) adsorption isotherm and a high temperature adsorption isotherm. Since the heat conductivity of the commonly used adsorbent is relatively low and both heating and cooling take a long time, the adsorption bed is relatively large, corresponding heating and cooling facilities are also to be provided, both energy consumption and investment are high, and the operation is more troublesome. In addition, substantial periodic changes in the temperature will affect the adsorbent life. Therefore, TSA is mainly used for the removal of trace impurities or impurities difficult to desorb, and TSA has the advantages of complete regeneration, high rate of recycling and less product gas loss, so it is still a widely applied method. However, the general TSA method has obvious disadvantages in the treatment of many gas separation conditions, such as industrial tail gas or waste gas, which are mainly reflected in several aspects as follows: first, the cycle period of the TSA method is long: since the heating and cooling process is relatively slow, which usually takes several hours or more than a day, so the adsorption time must be equal to or greater than the regeneration time to ensure that the bed adsorbate in an adsorption state is not penetrated at the end of regeneration. For this reason, the TSA process is not suitable for the working conditions with diffusion rate as the mechanism for adsorption, for example, the carbon molecular sieve (CMS) adsorbent is used to separate and extract nitrogen from air; second, the TSA method has a narrow scope of application: the TSA method is generally more suitable for the removal of trace impurities and the gas purification process, or occasions where it is difficult to regenerate completely by PSA depressurization or flushing. The scope of application is narrow, and the TSA method is mainly applied in several fields, comprising: removal or recycling of organic compounds and low concentration volatile organic compounds (VOC), clarification such as naphthalene removal from coke oven gas, recycling of vinyl chloride from PVC tail gas, removal or recycling of acidic components in gas (adsorption and clarification of removed or recycled sulfur dioxide (SO2), adsorption and clarification of removed or recycled NOX, low temperature clarification of low boiling point gas and the like), as well as drying and clarification of fluid, including clarification and drying in air, dehydration and drying of various materials and the like; third, the TSA method has high investment and high energy consumption: even if the TSA method is suitable for occasions where it is applied, the regeneration time is difficult to match with the adsorption time because regeneration requires higher temperature and energy consumption, resulting in relatively low regenerative efficiency; the long cycle period leads to large adsorption bed, large equipment volume and requirement for higher heat energy, so the investment of the TSA method is relatively high; fourth, the TSA method needs to choose an appropriate heat carrier: the new carrier generally needs to be introduced in the separation system, so that the TSA process becomes complex, and the application is limited. It is very important to choose the appropriate heat carrier. For example, the heat carrier fluid as regeneration gas shall contain no component to be desorbed, or common heated inert hot gas or nitrogen. Under conditions without common inert gas sources or nitrogen or conditions where these gas sources cannot be used as the regeneration gas, the clarified gas itself can be used as a regenerative carrier, but this will lead to a decrease in the yield of clarified gas or an increase in the circulating load of the TSA method. In addition, the flow rate of the regenerative heat carrier selected by the TSA method is relatively high, the operation cost will be further increased, particularly in the process of removal of trace impurities, a large number of heat carriers are used to heat a dead space in the adsorption bed, so that uneven temperature distribution and stagnation in the adsorption bed are increased, the turbulence phenomenon is serious, so that the efficiency of bed mass transfer is low. The higher the temperature requirement in the regeneration process, the greater the limitation of the heat carrier selected by the TSA method, the greater the required energy consumption, the lower the efficiency of the mass transfer process of the adsorption bed, and the greater the impact on yield; fifth, the heat carrier as the regeneration gas needs to be subsequently treated in the TSA method: for example, in the air-drying treatment, when regeneration is performed with hot nitrogen, the hot nitrogen in regeneration gas contains removed moisture and other impurities, forming the so-called “impure nitrogen”, which needs to be treated before recycling, and the greater the treatment capacity, the higher the cost of subsequent treatment. Sometimes impure nitrogen can be used directly as regeneration gas, but the service life of the adsorbent will be affected. Again, for instance, in the working conditions where many TSA methods are applied, the adsorbates are volatile organic compounds (VOCs). Since components of VOCs often contain alcohol, ether and ester components such as acetone which are easy to completely dissolve with vapor or form azeotrope, VOCs adsorbed by the adsorbents cannot be completely recycled or separated from the regeneration gas, so that the desorption gas meets the emission requirements, or the pure regeneration gas is recycled. Even if the subsequently added treatment system allows recycling or emission, other regeneration gases have to be used as heat carriers because of high investment and diseconomy, or the TSA method cannot be used to realize the desorption of VOCs; sixth, the application of the TSA method has certain potential safety hazards: for example, when a gas with more than 8-10% (v/v %) oxygen content, especially air, is used as the regeneration gas, and when the adsorbate components are volatile materials or easy to have oxidation reactions, if the regeneration temperature is improperly controlled, or when a sudden stop is encountered and the heat in the bed is not taken away in time, the commonly used adsorbent is activated carbon which will burn or explode, resulting in a great potential safety hazard; again, for instance, when VOCs or acid components are removed and recycled by the TSA method, the corrosivity of regeneration gas becomes very sensitive, especially under the condition where vacuum pumping (negative pressure) is required in order to make the adsorbent regenerate completely, the corrosion of acid gases to the vacuum pump or the corrosion of volatile components to the valve seal and the like will lead to leakage and air introduction, which will also cause serious potential safety hazards; seventh, the TSA method is not suitable for conditions with higher concentration of most adsorbates in the raw gas: this is due to the need for a large number of adsorbents or more adsorption beds standing side by side to match the time in the adsorption phase with the regeneration time, and the investment and operation costs are quite high; eighth, the service life of adsorbents in the TSA method is not long. During the running of the adsorption bed, the adsorbents shall have enough strength to reduce breakage and wear due to substantial periodic changes in the temperature within the bed. Too high temperature will also deteriorate or pulverize the adsorbents. In order to improve the regeneration efficiency of the TSA method and overcome the limitations of the adsorption-regeneration cycle application in the TSA method usually carried out only at normal pressure, if vacuum pumping and heating at the time of regeneration are considered, this process becomes a TPSA process, which is an improvement of the TSA method.

The TPSA separation method is a coupling between the TSA method and PSA method, namely the TPSA method dominated by the PSA method, and this method is essentially different from the TSA method. The TPSA method is generally adsorption at normal temperature and pressure. During regeneration, the temperature is raised, and the pressure is reduced to atmospheric pressure, even to negative pressure. The TPSA method can overcome some limitations in the regeneration process of the general TSA method, so that the energy consumption and regeneration time needed in the regeneration process of the TSA method are somewhat reduced, and then the purpose of matching with the adsorption time is achieved. However, since this TPSA method is still dominated by PSA, the regeneration process is mainly done by pressure swing rather than only by temperature swing, the technical bottlenecks of high energy consumption, high consumption of adsorbents, difficult matching of adsorption and regeneration time, narrow applications, need for selection of heat carrier as regeneration gas and the like existing in the TSA method are not fundamentally solved. Under some conditions, the application of the TPSA method can also bring the practical problems of difficult operation, increase in investment, short service life of adsorbents and the like.

Generally, the adsorption-separation method can solve some universal limitations and disadvantages of high energy consumption, low purity and the like in the conventional separation methods by properly coupling with the conventional power-wasting separation methods such as rectification, absorption and the like. However, the vast majority of such coupling processes still regulates or matches on the basis of the conventional separation methods, and they can neither fundamentally solve many problems in the conventional separation methods nor replace the basic role of the conventional separation methods. Meanwhile, the self-contained energy specific to raw gas cannot be fully used, or the application areas of general PSA or TSA or TPSA cannot be expanded.

The present invention emerges at the right moment under the above background.

### SUMMARY OF THE PRESENT INVENTION

Full Temperature Range-Pressure Swing Adsorption (FTrPSA) is a PSA-based method which, fully using the temperature and pressure of different raw gases as well as the differences in adsorption separation coefficients and physicochemical properties among all components in the raw gases at a temperature range of −80-200° C. and a pressure range of 0.03-4.0 MPa, regulates the adsorption or desorption regeneration in the PSA cycle process by coupling various separation methods, thus realizing the easy-to-match and easy-to-balance cyclic operation of adsorption and desorption regeneration in the process of PSA to separate, purify and clarify various mixed gases.

The method for gas separation, purification and clarification by FTrPSA of the present invention solves the difficult-to-match problem of adsorption and regeneration in the conventional adsorption field.

In order to solve the above problem, the present invention adopts the following technical solution:

A method for gas separation, purification and clarification by FTrPSA, comprising following procedures proceed in turn:

1) executing PSA concentration procedure: introducing raw gas into a PSA system; wherein the PSA system uses a multi-tower series or parallel connection process for alternate cyclic operation, with an adsorption temperature maintained at −80-200° C. and an adsorption pressure at 0.03-4.0 MPa; the raw gas is divided into two paths, intermediate gas and concentrated gas, which are separated for subsequent treatment; wherein, −80-20° C. is the intercooling & shallow-cooling temperature range suitable for the preparation of ultra-pure industrial gases, such as H2, O2, N2, Ar, CO and the like, recycling of C2 and above from refinery dry gas, low temperature methanol washing tail gas and the like, including purification of H2, CO and other industrial tail gases; 50-200° C. is the medium & high-temperature range suitable for recycling and clarification of effective components such as ethylene cracking gas, ethylene tail gas, coke oven gas, refinery dry gas, hydrogen-containing gas desulphurization and naphthalene removal, gas clarification, clarification of pharmaceutical tail gas and the like; 20-50° C. is the normal temperature section suitable for the purification and separation of gases with boiling points in the normal temperature section;

2) executing intermediate gas treatment procedure: wherein the intermediate gas is an unadsorbed gas in the PSA concentration procedure; which is discharged directly or stored, or refined in the refining procedure before being discharged or stored; and

3) executing concentrated gas treatment procedure: wherein the concentrated gas is the adsorbed gas in the PSA concentration procedure; the adsorbate component is further recycled or removed in the adsorbate recycling and removal procedure.

Further, before the PSA concentration procedure, the raw gas enters the pretreatment procedure for dedusting, preliminary concentration or preliminary purification, and a temperature of the raw gas is adjusted to −80-200° C.

Further, the raw gas is one or more of following components:

1) components with a low boiling point: hydrogen, nitrogen, oxygen, carbon monoxide, methane, argon and helium;

2) hydrocarbon components with a high boiling point: ethane, ethylene, propane, propylene, C4 and above;

3) oxygen-containing compound components volatile or easy to form azeotrope when meeting water: alcohols, ethers, ketones, esters, benzene and naphthalene;

4) water, carbon dioxide, ammonia and ammonia compounds, sulfur and sulfur compounds, oil and tar, starch, macromolecular organic compounds and proteins; and

5) mixed tail gas: ethylene cracking gas, ethylene tail gas, refinery dry gas, coke oven gas, hydrogen-sulfide-containing hydrogen-rich gas, rectisol tail gas, synthetic gas, purge gas from ammonia synthesis loop, methanol tail gas, overhead gas, carbon-monoxide-containing steelmaking tail gas, acetic acid tail gas, glycosylated tail gas, ethylene or propylene tail gas, polyolefin tail gas, chlor-alkali tail gas, biogas, natural gas, biomass gas, flue gas, volatile organic vapor, pharmaceutical tail gas and industrial purified gas or industrial tail gas.

Further, the pretreatment procedure comprises one or more of following operations:

1) executing preliminary impurity removal operation: wherein moisture, oil mist and impurity components which affect the PSA concentration procedure are removed preliminarily from the raw gas by means of drying, adsorption, condensation or scrubbing-absorption;

2) executing temperature regulating operation: wherein a temperature of the raw gas is regulated by heat exchange equipment and condensing equipment, and regulated within the operating temperature for the PSA concentration procedure; and

3) executing pressure regulating operation: wherein a pressure of the raw gas is regulated by pressure raising and reducing equipment and regulated within the operating pressure for the PSA concentration procedure.

Further, the preliminary impurity removal operation proceeds in a dust collector, water cooling tower, scrubber tower, condensing tower, rectification tower, PSA tower or primary adsorption tower.

Further, the heat exchange equipment in the temperature regulating operation is a heat exchanger or a heater.

Further, the condensing equipment in the temperature regulating operation is a water cooling tower, scrubber tower or condensing tower, and the raw gas is cooled by means of scrubbing or water cooling or condensation.

Further, the pressure raising and reducing equipment in the pressure regulating operation is a pressure reducer, throttle valve, blower or compressor.

Further, the PSA concentration procedure proceeds in the PSA system which consists of N adsorption towers, and N is a natural number greater than 1; wherein 1 to N−1 adsorption towers are in an adsorption state, and the remaining adsorption tower is in a regeneration state; the adsorption towers are formed by connection of the N towers in series or in parallel or a combination thereof for alternate cyclic operation.

Further, an adsorption mode of the adsorption tower is a concentration-based feed adsorption or staged adsorption or a combination thereof, depending on the comparison of the composition and pressure of raw gas with the composition, concentration and pressure of intermediate gas generated from the FTrPSA concentration procedure.

Further, a regeneration mode of the adsorption tower is one or more of evacuation, constant pressure, flushing and displacement gas, depending on the comparison of the composition and pressure of raw gas with the composition, concentration and pressure of intermediate gas generated from the FTrPSA concentration procedure.

Further, a filler in the adsorption tower is one or more of activated carbon, silica gel, activated aluminium oxide and molecular sieve, depending on the comparison of the composition and pressure of raw gas with the composition, concentration and pressure of intermediate gas generated from the FTrPSA concentration procedure.

Further, the refining procedure in the intermediate gas treatment procedure comprises one or more of catalytic oxidation reaction, catalytic reduction reaction, solvent absorption, membrane separation, PSA, TSA, rectification and cryogenic separation.

Further, a gas component from the refining procedure is discharged directly, stored or returned to the PSA concentration procedure for further adsorption treatment.

Further, the gas component from the refining procedure is regulated such that a temperature and a pressure of the gas component can reach an operating temperature and an operating pressure of the PSA concentration procedure before returning to the PSA concentration procedure.

Further, the adsorbate recycling and removal procedure comprises one or more of catalytic oxidation reaction, catalytic reduction reaction, solvent absorption, membrane separation, PSA, TSA, rectification and cryogenic separation.

Further, the concentrated gas is pressurized by the compressor or heated or cooled by the heat exchanger before entering the adsorbate recycling and removal procedure.

Further, the gas component from the adsorbate recycling and removal procedure is discharged directly, stored or returned to the PSA concentration procedure for further adsorption treatment.

Further, the gas component from the adsorbate recycling and removal procedure is regulated such that its temperature and pressure can reach the operating temperature and operating pressure of the PSA concentration procedure before returning to the PSA concentration procedure.

The method realizes the gradient utilization of self-contained energy (temperature and pressure) of the raw gas in the separation and clarification operations on the main body: for example, in the process of ethylene cracking gas separation, the temperature of the raw gas is 600-800° C., while the cryogenic rectification temperature of the separation unit as the main body is −110-−70° C., and the self-contained latent heat of the raw gas wastes huge energy and requires external energy. In the FTrPSA method, the main-body separation and clarification unit is the PSA concentration procedure, the operating temperature of the PSA concentration procedure is 60-200° C., the self-energy of the raw gas is greatly utilized and passed to all subsequent procedures through the energy cascade of the main-body separation and clarification unit, and the gradient utilization of energy in the gas separation process is truly realized.

The main-body separation and clarification unit in the method can determine the corresponding operating temperature and pressure of the main-body separation and clarification unit based on the differences of adsorption separation coefficients and physicochemical properties among all components in different raw gases within the temperature range of −80-200° C. and the pressure range of 0.03-4.0 MPa, for example, the raw gas is ethylene cracking gas which gives priority to the main components of ethylene, ethane, propylene and the like with higher boiling points, with H2, CH4, CO and the like with lower boiling points as supplements, thus the operating temperature of the main-body separation unit in the process of separation and clarification of ethylene cracking gas FTrPSA is controlled within 70-160° C.

The main-body separation and clarification unit in the present invention generates two paths of gas through medium & high-temperature or intercooling & shallow-cooling PSA concentration respectively on the raw gas dominated by components with higher boiling points or raw gas containing components with lower boiling points, i.e. intermediate gas of non-adsorbed phase, which contains components with lower boiling points, and concentrated gas of adsorbed phase, which contains components with higher boiling points, both of which enter the subsequent refining or recycling procedure formed by various separation methods to obtain the product gas or clarified gas or recycled components, so that the method for separation, purification and clarification of FTrPSA becomes a basic separation and clarification unit which replaces or partially replaces the conventional rectification, absorption and other separation methods, and expands the scope of the conventional PSA only as purification of end product gas.

The present invention expands the rationale of the conventional PSA adsorption-desorption regeneration cyclic operation process confined to regulation of pressure parameters, i.e., pressurization beneficial to adsorption and depressurization beneficial to desorption regeneration, into the balanced and adjustable cyclic operation between PSA adsorption and desorption regeneration that can be achieved by coupling adsorption and regeneration processes with other separation methods. Similarly, the rationale of the present invention expands the adsorption and desorption regeneration cyclic operations of the conventional TSA and TPSA separation process confined to the regulation of temperature and pressure factors, into the matching operations of regulating the adsorption and desorption regeneration cycle by other separation methods.

In the conventional PSA cyclic operation, adsorption proceeds at normal temperature usually. The lower the temperature and the higher the pressure, the better the adsorption is. However, the easier it is to adsorb, the more difficult it is to desorb in general. For clarification of some gases with lower concentration of adsorbate (such as less than 10%) or preparation of high purity gas and the like, adsorption is relatively difficult due to lower partial pressure of the adsorbate. The method determines different operating temperatures and pressures according to the physical properties of the adsorbate component of the raw gas and the adsorption capacity on different adsorbents, controls the adsorption depth and desorption regeneration, regulates the cyclic process of adsorption and desorption regeneration with the help of other separation and clarification methods used to solve the contradiction and technical bottleneck between adsorption and desorption regeneration in the conventional PSA cyclic operation.

The method can form a closed and complete separation and clarification system with the main-body separation and clarification unit, the refining procedure and the adsorbate recycling and removal procedure, wherein a product gas or qualified effluent gas, as well as the removal of impurities or the recycling of effective components can be obtained respectively from the subsequent refining unit and adsorbate recycling and removal procedures; if the tail gases produced in corresponding procedures still contain small amounts of effective components to be purified or recycled, they can be returned to the main-body separation and clarification unit for further treatment, so that the purity and recycling rate or impurity removal rate of the product gas or recycled components are greatly improved.

Since the main-body separation and clarification unit in the method plays the role of basic separation and clarification under the premise of gradient utilization of energy, the load and energy consumption in subsequent procedures of refining, purification, removal of impurities, recycling and clarification can be greatly reduced. For example, for removal of benzene and naphthalene from coke oven gas, the raw gas is separated into benzene and naphthalene-rich concentrated gas and intermediate gas (coke oven gas) enriched with sulfur and ammonia impurities after passing the main-body separation unit in the FTrPSA process. The concentrated gas enters the procedure of oil absorption-rectification and removal of benzene and naphthalene, so that the absorbent (oil) consumption, reflux ratio of rectification, energy consumption, treatment capacity and the like in the absorption-rectification procedure are greatly reduced, the negative effects of impurities such as sulfur and ammonia on the oil absorption-rectification separation by removal of benzene and naphthalene, as well as meaningless energy consumption and occupied effective space of separation equipment as a result of a large amount of non-condensable gases (pure coke oven gas consisting of methane, hydrogen and the like) which “play a bit role” in the process of oil absorption-rectification by removal of benzene and naphthalene are accordingly reduced, and then the equipment investment and production cost are greatly reduced, and the efficiency of separation, purification and clarification is improved.

Compared with the prior art, the present invention has the advantages that:

1) The present invention realizes the gradient utilization of self-contained energy (temperature and pressure) of the raw gas in the process of gas separation, purification and clarification the first time, especially for the separation, purification and clarification of hot mixed gas reactants produced in a gas phase reaction in petrochemical, coal chemical and other basic chemical industries, for example, ethylene cracking gas, synthetic gas, reformed gas and the like, so that the energy consumption in the process of gas separation, purification and clarification is substantially reduced;

2) The present invention determines the basic role of the method of FTrPSA gas separation, purification and clarification presented herein as a field of gas separation, purification and clarification for the first time, replaces or partially replaces the basic role of the conventional separation, purification and clarification methods such as rectification, absorption and extraction within the scope of separation, purification and clarification of raw gases involved, and widens the scope of adsorption separation applications such as conventional PSA, TSA or TPSA and the role of the conventional ultimate “purification” procedure;

3) The present invention presents new idea of regulating and controlling the balance and matching for realizing the cyclic operation of adsorption and desorption regeneration depending only on temperature and pressure in the conventional adsorption separation process by coupling with other separation, purification and clarification methods for the first time, solves the prominent contradiction between adsorption and desorption regeneration in the conventional PSA or TSA or TPSA separation process is solved, and further widens the substantive connotation, popularization and application of adsorption, separation, purification and clarification;

4) The present invention greatly widens the scope of application of a method for gas separation, purification and clarification, overcomes the raw gases difficult to treat or treated at a higher cost in the separation, purification and clarification techniques of the conventional rectification, absorption, membrane separation, PSA, TSA, TPSA, combination of various separation methods and the like, for example, the cryogenic rectification, separation and purification of ethylene cracking gas with huge energy consumption, higher self-contained temperature and a very wide range of boiling points in all components, which cannot be replaced or applied by other separation methods; for clarification for impurities removal, such as medical tail gas with trace impurities (VOC) removed, coke oven gas with benzene, naphthalene and other impurities with high concentrations and the like, none of the conventional absorption, extraction, PSA and the like can be applied, the application of TSA is limited due to short service life of adsorbents, long time for removal of impurities, excessive investment and the like because the regeneration of TSA requires high temperature, heat carrier and the like; for the preparation of some high purity gases, the present invention makes up for the gap that no clarification method with better technical and economic performance is available except for cryogenic (ultra cryogenic rectification) separation with huge energy consumption, as well as sequentially catalytic adsorption clarification and separation of noble metal membrane with extremely high costs;

5) The present invention can form a closed and complete separation and clarification system with the main-body separation and clarification unit and the ultimate procedures of purification, removal of impurities and recycling, wherein a product gas or qualified effluent gas, as well as the removal of impurities or the recycling of effective components can be obtained respectively from the subsequent refining unit and the impurities removal, clarification and recycling unit; if small amounts of effective components to be purified or recycled are still contained in the tail gas produced in corresponding procedures, the tail gas can be returned to the main-body separation and clarification unit for further treatment, so that the purity of product gases or recycled components and the recycling rate or impurity removal rate are greatly improved;

6) As the main-body separation and clarification unit in the method has the effects of basic separation and clarification on the premise of gradient utilization of energy, the load and energy consumption in the subsequent procedures of refining, purification, impurities removal, recycling and clarification can be greatly reduced. For example, for removal of benzene and naphthalene from coke oven gas, the raw gas is separated into benzene and naphthalene-rich concentrated gas and intermediate gas (coke oven gas) enriched with sulfur and ammonia impurities after passing the main-body separation unit in the FTrPSA process. The concentrated gas enters the procedure of oil absorption-rectification and removal of benzene and naphthalene, so that the absorbent (oil) consumption, reflux ratio and energy consumption of rectification, treatment capacity and the like in the absorption-rectification procedure are greatly reduced, the negative effects of impurities such as sulfur and ammonia on the oil absorption-rectification separation by removal of benzene and naphthalene, as well as meaningless energy consumption and occupied effective space of separation equipment as a result of a large amount of non-condensable gases (pure coke oven gas consisting of methane, hydrogen and the like) which “play a bit role” in the process of oil absorption-rectification by removal of benzene and naphthalene are accordingly reduced, and then the equipment investment and production cost will be accordingly reduced, and then the equipment investment and production cost are greatly reduced, and the efficiency of separation, purification and clarification is improved;

7) The main-body separation and clarification unit generates two paths of gas through medium & high-temperature or/and intercooling & shallow-cooling PSA concentration process respectively on the raw gas dominated containing components with higher boiling points or/and raw gas containing components with lower boiling points, i.e. intermediate gas of non-adsorbed phase, which contains components with lower boiling points, and concentrated gas of adsorbed phase, which contains components with higher boiling points, both of which enter the subsequent refining or recycling procedure formed by various separation methods to obtain the product gas or clarified gas or recycled components, the problem of efficiency affected by mutual interference of different components in their processes of separation, purification and clarification is avoided, so that the method for separation, purification and clarification of FTrPSA becomes a basic separation and clarification unit which replaces or partially replaces the conventional rectification, absorption and other separation methods, and expands the purification effect of the conventional PSA only as the end product gas;

8) Especially in the preparation and clarification of high purity gas of low temperature separation, the main-body separation and clarification unit effectively reduces the energy consumption of the conventional cryogenic or ultra-cryogenic rectification, low temperature adsorption (TSA), high pressure adsorption and other methods, and provides a way of very high technical and economic values for the field of preparation and clarification of high purity gas.

### BRIEF DESCRIPTION OF THE DRAWINGS

FIG. 1 is a flow diagram of a method of the present invention applied to clarification of benzene and naphthalene removal from coke oven gas;

FIG. 2 is a flow diagram of the method of the present invention applied to separation and purification of ethylene cracking gas;

FIG. 3 is a flow diagram of the method of the present invention applied to removal of H2S from a hydrogen-rich gas source;

FIG. 4 is a flow diagram of the method of the present invention applied to preparation of ultra-pure hydrogen by methanol cracking;

FIG. 5 is a flow diagram of the method of the present invention applied to technique of pure oxygen preparation;

FIG. 6 is a flow diagram of the method of the present invention applied to recycling of C2 and above from refinery dry gas;

FIG. 7 is a flow diagram of the method of the present invention applied to recycling of ethylene and propylene from polyolefin tail gas;

FIG. 8 is a flow diagram of the method of the present invention applied to recycling of C2 and above from low temperature methanol washing tail gas;

FIG. 9 is a flow diagram of the method of the present invention applied to medical tail gas clarification VOCs;

FIG. 10 is a flow diagram of the method of the present invention applied to methane preparation from natural gas.

### DETAILED DESCRIPTION OF THE PREFERRED EMBODIMENT

In order to enable those skilled in the art to have a better understanding of the method, a clear and complete description of the technical solution in the embodiments will be given combined with the figures in the embodiments of the method.

### Embodiment 1

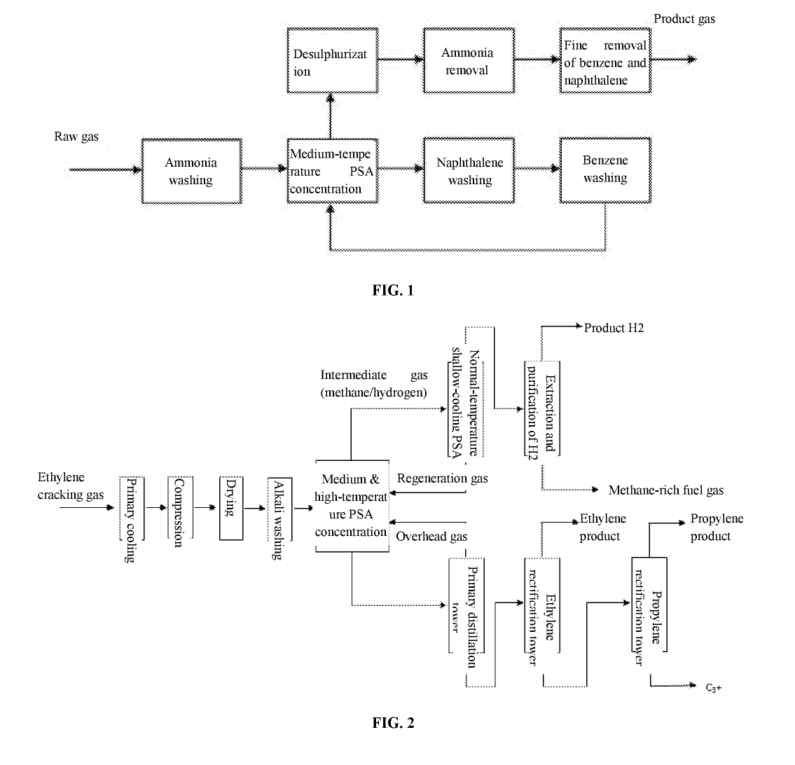

As shown in FIG. 1, a method for gas separation, purification and clarification by FTrPSA, applied to the clarification of coke oven gas by removal of benzene and naphthalene, comprises the following operations in turn:

(1) Pretreatment procedure: ammonia washing is performed, the coke oven gas at 650-800° C. produced from a coke oven has the temperature of 60-120° C. and pressure of 0.3-2.8 MPa after ammonia washing, 70-75% of coal tar and a part of benzene in the coke oven gas are condensed down, the tar drops from about 80-120 g/m3 to about 35-40 g/m3, benzene drops from 30-45 g/m3 to below 3-3.5 g/m3, and naphthalene drops from 8-12 g/m3 to below 0.8 g/m3.

(2) Medium-temperature PSA concentration procedure: the coke oven gas after ammonia washing enters the PSA system for adsorption, 8 adsorption towers are connected in series for alternate cyclic operation at the operating temperature of 60-120° C., the intermediate gas of non-adsorbed phase enriched with sulfur and ammonia with benzene, naphthalene and tar roughly removed flows from the top of the adsorption towers for the next step of clarification treatment, the tar contained therein drops to below 20-30 mg/m3, benzene drops to below 20-30 mg/m3, and naphthalene drops to below 0.1 g/m3; a concentrated gas with the concentration of benzene and naphthalene of 55-60% is obtained by desorption regeneration, and benzene and naphthalene in the concentrated gas are recycled.

(3) Intermediate gas treatment procedure: desulphurization, ammonia removal, and fine removal of benzene and naphthalene are carried out in turn:

a. Desulphurization: apply the wet oxidation process by using the self-contained ammonia in the coke oven gas as the absorbent and HPF as the catalyst, with the reaction temperature held at 22-30° C. First, acid components such as hydrogen sulfide (H2S) in the intermediate gas (coke oven gas) are converted into ammonium bisulfate and other acid ammonium salts, then oxygen in the air is transformed into elemental sulfur (S) by oxidation, so that the H2S removal efficiency in coal gas is up to above 90%; the desulphurized gas with hydrogen sulfide content below 15 mg/m3 is obtained after desulphurization;

b. Removal of ammonia: ammonia in the desulphurized gas is absorbed in an acid pickling tower by cyclic spraying of ammonium sulfate mother liquor, a deaminized gas supplied to the next procedure is obtained after separation of acid mist from the desulphurized gas coming out of the acid pickling tower, the unsaturated ammonium sulfate mother liquor from the acid pickling tower is supplied to a crystallizing tank in an evaporative crystallization portion, where vacuum evaporation, concentration and crystallization are carried out, so that ammonium sulfate crystalline particles constantly grow up through cyclic concentration of the mother liquor, the grown ammonium sulfate crystalline particles are pumped to a feed tank, and a ammonium sulfate product is obtained through centrifugal separation and drying;

c. Fine removal of benzene and naphthalene: the deaminized gas obtained from the ammonia removal procedure is treated by the one-time solid adsorption process, trace amounts of benzene and naphthalene are further removed to less than 1 ppm, the deaminized gas enters an adsorption bed, and the product coal gas supplied to the outside is obtained after complete adsorption; adsorbents are adsorbing materials for one or more composite filling beds, both adsorption towers are used for continuous operation, one is in the adsorption stage, and the other is in the adsorbent replacement or off-line regeneration stage.

(4) Concentrated gas treatment procedure: naphthalene washing and benzene washing proceed in turn:

a. Naphthalene washing: rich naphthalene and a part of benzene in the concentrated gas are absorbed by the naphthalene washing process with oil, the concentrated gas from the bottom of the absorption tower enters the bottom of two naphthalene scrubber towers, after washing by 55-57° C. benzene washing rich oil sprinkled down from the tower top, and a naphthalene washing gas with the naphthalene content decreasing from 2.5-4.5 g/m3 to 0.5 g/m3 is obtained;

b. Benzene washing: the naphthalene washing gas produced from the naphthalene washing procedure is absorbed by the cyclic washing oil process at 27-30° C., the naphthalene washing gas cooled to 25-27° C. via a final cooler passes two benzene scrubber towers in turn, benzene is generally reduced from 35-40 g/m3 to 2.0-3.0 g/m3, and the rest tail gas components containing a small amount of benzene and naphthalene are returned to the medium-temperature PSA concentration procedure for recycling.

In this embodiment, the ammonia washing procedure reduces the temperature of 120 km3/h raw coal gas from 650-800° C. to 60-120° C., the cooled raw coal gas enters the PSA system consisting of valves, pipeline assembly and 4 absorption towers connected in parallel for medium-temperature PSA for concentration at the operating temperature of 60-120° C., the desorption effect is good, the bed can be regenerated quickly, and the service life of the bed is greatly increased. After concentration, the intermediate gas with low contents of benzene and naphthalene and the concentrated gas with high contents of benzene and naphthalene are obtained. After passing the naphthalene washing and benzene washing procedures in turn, the concentrated gas flows back to the medium-temperature PSA concentration procedure, and the product gas can be obtained after the intermediate gas enters the procedures of desulphurization, ammonia removal and fine removal of benzene and naphthalene in turn; medium-temperature PSA is carried out without reducing the temperature to below 22-25° C. in this embodiment, and the sensible heat of gas is fully utilized; the medium-temperature PSA method is used in this embodiment for the concentration treatment of benzene, naphthalene and tar directly on the coal gas cooled through ammonia washing, so that the raw coal gas is separated into regeneration gas (concentrated gas) with a high concentration of benzene naphthalene tar, sulfides, ammonia and the like not adsorbed, and coal gas (intermediate gas) with trace benzene and naphthalene (dominated by trace naphthalene); meanwhile, the concentrated gas rich in benzene, naphthalene and the like enters the conventional work section of oil washing and removal of benzene and naphthalene, so that the efficiency of removal of benzene and naphthalene is greatly improved due to concentration increase (partial pressure increase) of oil washing and removal of benzene and naphthalene, the treatment capacity is also greatly reduced, and then the circulation of oil washing absorbent, consumption, energy consumption and other impurity interferences are also greatly reduced. At this time, the integrated process of side line naphthalene extraction from a benzene scrubber tower can also be used, which will further reduce the equipment investment and cost for removal of benzene and naphthalene, and a small amount of unabsorbed regeneration tail gas containing benzene, naphthalene and the like can be returned to the medium-temperature PSA procedure for further recycling treatment; after medium-temperature PSA and removal of benzene and naphthalene, the intermediate gas enters the conventional desulphurization and ammonia removal procedure, so that the negative effects of more impurities such as benzene, naphthalene and tar on its load and efficiency of desulphurization and ammonia removal are avoided, the load of fine removal of trace benzene and naphthalene in the coal gas is also relieved, the efficiency of fine removal is improved, and at this time, one-time fixed adsorption can be used for fine removal of benzene and naphthalene to obtain the qualified product coal gas. Since most of the impurities are firstly removed by concentration in the medium-temperature PSA concentration procedure, the load of back end treatment is reduced, the back end procedure of naphthalene and benzene blockage is avoided, and the production smoothness is improved; the procedures of primary cooling and tar precipitation in the conventional process are omitted in the whole process, the annual yield of crude benzene, naphthalene solvent oil or benzene washing and oil washing can be increased by more than 1000 tons with consumption reduced, the concentrated gas flows back to the medium-temperature PSA concentration procedure after the naphthalene washing and benzene washing procedures in turn, its effective gas is returned to the front-end medium-temperature PSA concentration procedure for recycling again, and therefore, the coal gas, naphthalene and benzene in this method can all reach an extremely high recycling rate; the oil containing impurities in the intermediate gas which enters the desulphurization procedure through the medium-temperature PSA concentration procedure are reduced, so that catalyst poisoning can be prevented, the consumption of expensive desulphurization catalyst is reduced, the quality of sulphur products will also be improved significantly, with the purity up to more than 98%; the ammonia removal procedure is arranged after the procedures of benzene and naphthalene concentration and desulphurization; since the tar content in the coal gas is obviously reduced, the amount of acid tar produced in the ammonia removal procedure can be drastically reduced compared with the conventional process, the acid tar treatment cost is accordingly reduced, which is conductive to environmental protection, the impurity content in ammonium sulfate is reduced, and the quality of the ammonium sulfate product is improved.

This embodiment has the advantages that the adsorbent life of the medium-temperature PSA tower is long, the energy consumption and cost for device operation are low, the phenomenon that benzene and naphthalene easily block the adsorption tower is avoided, the precision of removal of naphthalene and benzene is high, the yield of products and by-products is high, and catalyst poisoning is prevented.

### Embodiment 2

As shown in FIG. 2, a method for gas separation, purification and clarification by FTrPSA, applied to the separation and purification of ethylene cracking gas, comprises the following operations in turn:

(1) Pretreatment procedure: primary cooling, compression, drying and alkali washing are carried out in turn, the temperature is reduced to 80-200° C., the pressure is adjusted to 0.3-2.8 MPa, and heavy oil and heavy hydrocarbon are removed during primary cooling.

(2) Medium & high-temperature PSA concentration (procedure): the pretreated raw gas enters the PSA system for PSA, the PSA system is formed by connecting 10 adsorption towers in series for alternate cyclic operation at the operating temperature of 80-200° C. and pressure of 0.3-2.8 MPa, intermediate gas and concentrated gas are obtained, the intermediate gas flows out from the top of the adsorption tower, the intermediate gas is mainly a non-adsorbed phase consisting of H2, CO, methane, CO2 and the like which are nonpolar with lower boiling points in the cracking gas, and C2 and above (including ethylene, ethane, propylene and the like) with higher boiling points and escape content of about 1-10% are carried; the concentrated gas is the desorbed gas of the adsorbed enriched C2 and above.

(3) Intermediate gas treatment procedure: the intermediate gas enters the normal temperature or shallow cooling PSA procedure to further adsorb C2 and above, the non-adsorbed gas obtained in this procedure is a methane hydrogen fuel gas rich in components such as H2, CO and CH4, and the gas is direct burnt or supplied to the hydrogen extraction procedure of methane hydrogen PSA to obtain pure hydrogen and methane-rich fuel gas; the desorbed gas obtained in the adsorbed phase of this procedure is returned to the medium & high-temperature PSA concentration procedure after pressurization and heat exchange.

(4) Concentrated gas treatment procedure: the concentrated gas enters the conventional ethylene and propylene rectification system after pressurization and heat exchange: the concentrated gas firstly enters the primary distillation tower and flows directly into the ethylene rectification tower from the top of the primary distillation tower, ethane products with purity of 99% are produced from the bottom of the ethylene rectification tower, and ethylene products with content of 99% are produced from the top of the tower; the heavy components flowing out from the bottom of the primary distillation tower reenter the propylene rectification tower, propylene products with purity of 99% (VN, the same below) are produced from the top of the tower, and other components such as C3 and above flow out from the bottom of the tower.

For the first time, this embodiment presents the medium & high-temperature PSA concentration and extraction of ethylene and propylene on the ethylene cracking gas with the high temperature of 700-900° C. after primary cooling and other pretreatment procedures within the medium & high-temperature range of 80-200° C. Compared with the conventional condition of −110-−70° C. in the cryogenic rectification series of main-body separation procedure carried out after quenching and other pretreatment procedures, the FTrPSA gas separation and clarification method is better for the gradient utilization of the self-contained energy of ethylene cracking gas, the temperature of the concentrated gas after the medium & high-temperature PSA concentration procedure of FTrPSA is consistent with the operating temperature of the system entering the subsequent ethylene propylene rectification, so that the temperature throughout the separation and clarification process does not changed much, and the energy consumption is very low; ethylene and propylene concentration treatment in the FTrPSA separation and clarification method is firstly carried out on the ethylene cracking gas cooled and pretreated directly by primary cooling, pressurization, alkali washing, drying and the like by using the procedure of medium & high-temperature PSA concentration, so that the raw ethylene cracking gas is separated into the regeneration gas (concentrated gas) containing C2 and above (ethylene, propylene and the like) with higher concentrations and higher boiling points, non-adsorbed H2, CO, methane, CO2 and the like dominated by low boiling point components, as well as intermediate gas (methane hydrogen) of entrained trace ethane, ethylene and other components; meanwhile, the concentrated gas rich in ethylene and propylene enters the conventional work section of ethylene and propylene rectification, so that the efficiency of ethylene and propylene rectification separation is greatly improved due to the increased concentration (increased partial pressure) of ethylene and propylene rectification, the treatment capacity is also greatly reduced, the reflux ratio, energy consumption and others such as “playing a bit role” and interference of methane hydrogens such as low boiling point components H2 and methane are also greatly reduced, a small amount of rectification tail gas of methane hydrogen which “plays a bit role” can be returned to the medium-temperature PSA procedure for further recycling treatment, or directly burnt as fuel gas, or directly supplied into the PSA procedure of hydrogen extraction for further extraction of H2; after the medium-temperature and medium & high-temperature PSA concentration procedures, the intermediate gas (methane hydrogen) containing 1-10% C2 and above enters the procedure of normal temperature or shallow cooling PSA extraction of H2, so that the negative effects of more low boiling point components which “play a bit role” and the like on the load and efficiency of ethylene and propylene production by separation of ethylene cracking gas are avoided, at the same time, the load for ethylene and propylene refining from ethylene cracking gas is reduced, and the separation efficiency is improved. At this time, the desorbed gas in the procedure of normal temperature or shallow cooling PSA extraction of H2 can be returned to the medium & high-temperature PSA concentration procedure for further recycling treatment, so that the whole process of FTrPSA separation and clarification forms a complete closed system.

The adsorbent used in the medium & high-temperature PSA concentration procedure of this embodiment has a long life, the principle is PSA without periodic temperature change and desorption is complete without long-term residues of harmful substances to the adsorption bed, so the adsorbent used in the medium & high-temperature PSA concentration procedure has a long life; the system operating load of subsequent ethylene and propylene rectification of the concentrated gas is further reduced, and the load and extraction energy consumption for further H2 extraction from the intermediate gas, i.e. methane hydrogen, are also lightened. The methane hydrogen produced by the conventional cryogenic rectification process has a low temperature and is suitable for cryogenic separation. But since a balance between a large amount of hydrogen, a small amount of methane and CO in methane hydrogen has been formed, and it is difficult to extract H2 with higher purity by the conventional cryogenic separation process. In the FTrPSA gas separation method, the temperature of the intermediate gas (methane hydrogen) generated from medium & high-temperature PSA concentration procedure is relatively high, the adoption of normal temperature or shallow cooling PSA hydrogen extraction can save not only part of latent heat (self-contained energy) of methane hydrogen but also obtain the product hydrogen with higher purity, the desorbed gas is pressurized and returned to the front-end medium & high-temperature PSA concentration procedure, so that the yield of H2 and the yield of ethylene and propylene are improved; the ethylene and propylene product has high purity and yield, since the concentrated C2 and above are separated from the purified C2 and above, most ethylene and propylene are firstly enriched from the concentrated gas (ethylene and ethane rich gas) in the medium & high-temperature PSA concentration procedure, a small amount of ethylene and propylene is recycled again from the intermediate gas (methane hydrogen) in medium & high-temperature PSA through normal temperature or shallow cooling PSA and the like, then higher purity and yield of both of the ethylene and propylene products can be guaranteed on the premise of higher recycling rate of H2, the purities of both are 299%, and the yield is 297-99%; the concentrated gas enriched with ethylene and propylene produced from the medium & high-temperature PSA concentration procedure is directly supplied into the conventional ethylene and propylene rectification procedure, so that the cryogenic rectification steps such as demethanizing tower and deethanizing tower of the conventional cryogenic rectification are saved, the energy consumption and equipment investment are greatly reduced, and the stability of ethylene and propylene production is further improved.

### Embodiment 3

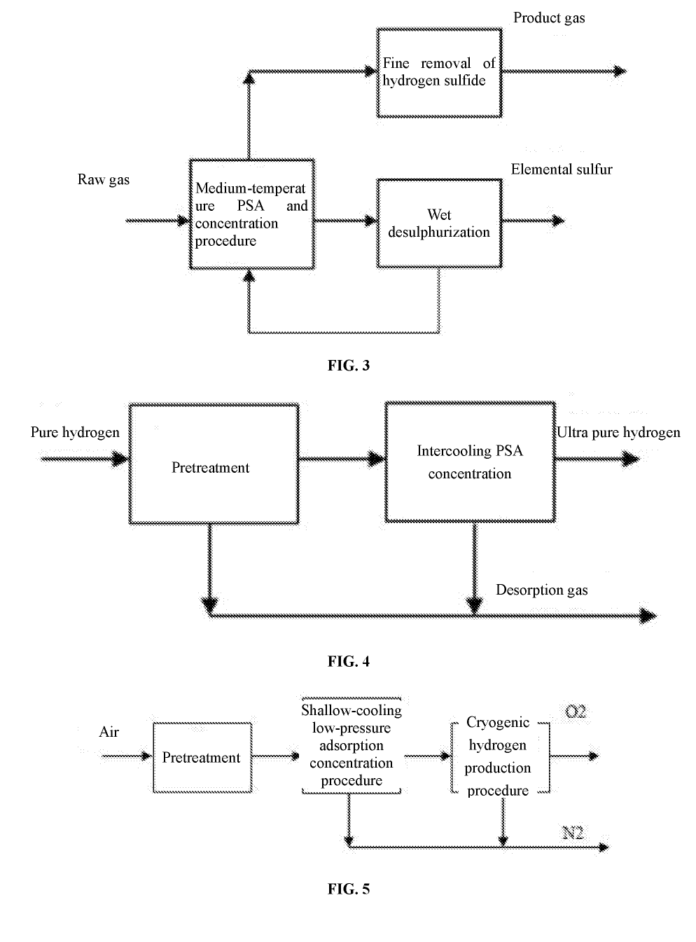

As shown in FIG. 3, a method for gas separation, purification and clarification by FTrPSA, applied in the field of H2S removal from hydrogen rich gas source, comprises the following operations in turn:

(1) Medium-temperature PSA concentration procedure: a hydrogen rich gas with flow rate of less than 1000-10000 standard volume/hour, temperature of 50-150° C. and H2S concentration of 4-10% is used as the raw gas supplied into the PSA system via an air intake duct for PSA and alternate cyclic operation by the process of connecting 6 towers in series or in parallel at the operating temperature of 50-150° C., the intermediate gas containing a small amount of hydrogen sulfide is obtained after the adsorption is completed, and a concentrated gas rich in hydrogen sulfide is obtained after vacuum pumping and desorption.

(2) Intermediate gas treatment procedure: the intermediate gas enters the subsequent hydrogen refining procedure, i.e. the fine H2S removal procedure, a dry method consisting of two one-time adsorption towers is used for fine desulphurization hydrogen treatment, with zinc oxide as the desulphurizing agent, the product hydrogen (H2S≤0.1 ppm) flows out from the tower top into the product gas pipeline after adsorption in the H2S fine removal procedure for a certain period of time, the adsorbent is regenerated off line or replaced with a new adsorbent as the adsorbent of H2S is transferred out from the one-time adsorption tower, the regeneration gas body still contains a certain amount of H2S which is returned to the medium-temperature PSA concentration procedure after pressurization to continue with recycling treatment; at this time, the other tower is in the adsorption stage, and continuous production is realized.

(3) Concentrated gas treatment procedure: the concentrated gas directly enters a sulfur production device for wet desulphurization, enters the first-stage desulphurization tower from the lower part of the desulphurization tower and comes into countercuttent contact with desulphurizing solution sprinkled on the packing surface at the tower top, H2S is absorbed by the desulphurizing solution, the desulphurization rich solution which absorbs H2S flows out from the tower bottom, then the desulphurization rich solution is fed into a self-priming air-jet regeneration tank via a rich solution pump, at the same time, the desulphurization solution is regenerated by the automatically draw-in air, the air comes out with the desulphurization solution from the ejector tail pipe and comes into countercuttent contact with the desulphurization solution again from bottom to top; under the action of the catalyst, the sulfides and sulfohydrates in the solution are oxidized into elemental sulfur and brought by ascending air to the upper level of the regeneration tank to form sulfur foam, the regenerated and oxidized solution flows from the bottom of the regeneration tank into a lean solution tank using the static pressure difference, drawn out by a lean solution pump and pumped into the desulphurization tower for recycling; the sulphur foam generated in the process of oxidation and regeneration of desulphurization solution automatically flows from the overflow weir of the regeneration tank into the foam tank by using the potential difference, and the sulphur foam is pumped by the foam pump into a sulfur melting device; while the sulphur product is obtained, the separated desulphurization solution flows back into the desulphurization solution to reduce the loss of soda ash and catalyst and maintain the water balance of the desulphurization system. The desulphurization rate is greater than 95-96%.

In this embodiment, since most of hydrogen sulfide is removed from the intermediate gas, the sulfur content is low (H2S≤50 ppm), but the sulfur concentration requirement of the next work section is not satisfied, and fine hydrogen sulfide removal treatment is also required. For processes with low flow rate of raw gas and less sulfur content, a dry process consisting of two one-time absorption towers is used to perform fine hydrogen sulfide removal treatment, and zinc oxide is used as the desulphurizing agent. In general, H2S adsorption by zinc oxide can be carried out at normal temperature, and the adsorption reaction rate is significantly accelerated as the temperature rises. The temperature of intermediate gas flowing out in the medium-temperature PSA concentration procedure of the previous work section is 50-150° C., which is more beneficial to the absorption of H2S, and the sensible heat of gas is fully used. This desulphurization method has high precision of desulphurization and makes the discharged sulphur<0.1 ppm, the process is simple, the flow is short, no reduction is needed after filling, nitrogen can be used directly after nitrogen displacement and heating, and the method is suitable for working conditions with less S content; the sulphur content of the concentrated gas meets the requirement of the wet desulphurization and sulphur production system, and the concentrated gas can be directly fed into the wet desulphurization and sulphur production device.

This embodiment completely solves the technical problem that wet desulphurization and sulfur production with higher concentration and dry fine removal are combined in series and unable to match up with each other as a result of great fluctuations of the H2S concentration in the raw gas in the conventional H2S removal and clarification process, enables H2S in the hydrogen rich raw gas of low-pressure low-concentration H2S to concentrate to meet the requirements of the wet desulphurization and sulphur production device in a single step, effectively reduces the load of the deep H2S removal process in the next step, thus reducing the energy consumption and material consumption of the entire desulphurization device, and the problems of high circulation flow, great loss, high energy consumption and long flow of chemical solvents caused by the large amount of gas in the prior art are solved; the contradiction between adsorption and regeneration in the conventional PSA desulphurization process is solved, wherein the adsorbent in the medium-temperature PSA concentration procedure has a long life, the common dry desulphurizing agent is difficult to regenerate or cannot be regenerated, with environmental pollution caused by the desulphurization wastes and other problems; the operating temperature of the medium-temperature PSA concentration procedure is 50-150° C., the sensible heat energy of the raw gas is utilized, namely that this embodiment fully explores the energy gradient utilization rate of the raw gas and reduces the energy consumption. Other techniques require a temperature drop of the raw gas to the normal temperature of 20-45° C., which causes a great sensible heat waste of the raw gas; the technological operation is simple, and the process is stable and reliable. Since frequent replacement of the adsorbent is not required, the PSA technique is adopted in the whole concentration and fine removal process, so that continuous production and fully automated operation are possible; the medium-temperature PSA concentration system is easy to regenerate, and H2S can be concentrated and easy to regenerate due to the adoption of FTrPSA technology; the method solves the problems of high energy consumption, difficult regeneration gas source and the like as a result of the fact that steam or other high temperature gases need to be input for regeneration in the prior art. In particular, when a small amount of elemental sulfur is deposited on the adsorbent surface, the temperature for TSA regeneration needs to be up to about 400° C. Furthermore, the adsorption bed suffers too much changes in the adsorption and regeneration temperature which drops from the high regeneration temperature to the normal temperature, the adsorbed H2S is difficult to completely desorb so that the adsorption efficiency is greatly reduced, the service life of adsorbent is also shortened because the corresponding temperature differences vary greatly in the adsorption regeneration process, and the FTrPSA method nicely solves the problem of short service life of the adsorption bed; the raw gas is separated into two paths in the medium-temperature PSA concentration procedure, one is the intermediate gas with a low H2S content which is suitable for the refining procedure, the other is the concentrated gas with a high H2S content suitable for the wet desulphurization procedure, thus changing the problems such as low efficiency of fine removal caused by the conventional sequential (connection in series) mode of “rough removal first, followed by fine removal” in the former desulphurization process which tends to bring the absorption liquid, catalyst and other new components used in the rough removal system into the fine removal system, the operating load of fine removal and wet desulphurization in the FTrPSA method is also further reduced, and the energy consumption is saved; for the condition of lower concentration of H2S contained in the raw gas, the non-absorbent material produced in the medium-temperature PSA concentration procedure is directly a qualified H2 product without undergoing the fine removal procedure; when the fine removal procedure is needed, the desorbed gas produced by its fine removal procedure can still be returned to the wet desulphurization procedure, so that the recycling rate of H2S is greatly increased to more than 93-96%.

### Embodiment 4

As shown in FIG. 4, a method for gas separation, purification and clarification by FTrPSA, applied in the field of clarification and extraction of ultra-pure hydrogen with pure hydrogen prepared by methanol cracking as a raw gas, comprises the following operations in turn:

(1) Preparation of pure hydrogen by methanol cracking: a. methanol conversion and hydrogen production: methanol and water are mixed, superheated and acts through a catalyst while a catalytic cracking reaction and a carbon monoxide conversion reaction occur, and a mixed gas of hydrogen (H2) and carbon dioxide (CO2) is finally generated; after heat exchange, condensation and separation of the mixed gas after the reactions, a reforming gas containing 74% hydrogen, 24.5% CO2 and 0.5% CO is obtained; b. hydrogen purification procedure: the reforming gas from the methanol conversion procedure is fed into the normal-temperature PSA device, the gas enters from the bottom of the adsorption tower for adsorption, the adsorption pressure is 2.0-3.0 MPa, and the operating temperature is the normal temperature; the hydrogen with a content of 98-99% is obtained after separation and purification; the hydrogen is obtained by hydrogen production and purification from methanol in this procedure, small amounts of impurities are mainly CO, CO2, water, oxygen-containing hydrocarbon (methanol) and N2, wherein N2 is the residue in the pipeline or equipment after device purging, the water and oxygen-containing hydrocarbons are the residues of unreacted conversion, and CO and N2 are the impurities most difficult to completely remove in the hydrogen purification procedure. Since all the relative separation coefficients of CO, N2 and H2 at normal temperature are less than 3 and easy to reach the equilibrium value, it is difficult for the conventional PSA method to further purify and remove small amounts of CO and N2, and the 98-99%, hydrogen enters the FTrPSA system for purification as the raw gas.

(2) Pretreatment procedure: small amounts of impurities such as N2, CO, water and oxygen-containing hydrocarbons contained in raw gas are adsorbed; this procedure is for one-time solid adsorption, containing two adsorption towers, and one of the two adsorption towers is always undergoing adsorption or regeneration; the raw gas enters from the tower bottom, the relatively pure hydrogen after adsorption and impurity removal is discharged from the top of the absorption tower as the raw hydrogen supplied into the next procedure, and the adsorbent is regenerated off line or replaced with a new adsorbent as the adsorbent is transferred out from the adsorption tower in a saturated adsorption state; small amounts of water, oxygen-containing hydrocarbons, CO2, CO and the like are further removed in this procedure to prevent icing or local aggregation of oxygen-containing hydrocarbons in intercooling PSA (“IncPSA”, the same below) from affecting the stable operation of the IncPSA system.

(3) IncPSA ultrapurification procedure: the pretreated and clarified hydrogen enters from the bottom of the adsorption tower in the IncPSA system for further adsorption and purification, the adsorption pressure is 2.0-3.0 MPa, and the operating temperature is −80-−10° C.; the adsorbed N2, CO, water, oxygen-containing hydrocarbons and other impurities are desorbed and regenerated by flushing and depressurization, discharged from the bottom of the adsorption tower and fed into the desorbed gas tank; after adsorption, an ultra-pure hydrogen product gas with the purity of above 99.9999% (6N) is obtained at the pressure of 2.0-3.0 MPa and temperature of −80-−10° C., discharged from the top of the adsorption tower and supplied for use.

Using the super adsorption performance of adsorbents for a small amount of impurity components such as water and oxygen and nitrogen hydrocarbons within the intercooling temperature range (−80-−10° C.), this embodiment avoids the technical problems of cryosorption, deep adsorption and difficult regeneration, and the technical problem that it is difficult for the conventional PSA to directly treat a small amount of impurities or impurities with extremely low partial pressure. This embodiment partially realizes the gradient utilization of the self-cooling capacity of raw gas, which enables the cyclic operations of adsorption and regeneration of the IncPSA purification system based on PSA in this method. A small amount of impurities contained in the hydrogen as a raw gas after clarification is removed in the procedure, so that the ultra-pure hydrogen product with the purity of 99.9999% (6N) is obtained, and the method also solves the technical bottleneck in the ordinary PSA method that the maximum product purity is 99.999% (5N) as a result of being difficult to separate and remove from hydrogen because a small amount of impurity components is in equilibrium with hydrogen; the regeneration process is completed by flushing and depressurization, without such as cryogenic adsorption and normal-temperature high-pressure adsorption which are, in essence, a heating process with the heat carrier that must be selected for TSA as the regeneration gas, and then new impurity components will not be introduced into the purification system, as in the case of cryogenic adsorption or normal-temperature high-pressure adsorption based on TSA, causing not only an increase in the load and treatment difficulty of the purification system, but also a great increase in energy consumption and an impact on the quality of ultra-pure hydrogen. Therefore, this embodiment greatly reduces the energy consumption of regeneration. Compared with the palladium membrane method which requires raw gas of pure hydrogen with higher temperature and pressure, and the lower temperature and higher pressure required by ultra-pure hydrogen product gas, the FTrPSA purification method has consistent requirements for temperature and pressure of raw gas and product gas, so that the energy consumption in the purification process is greatly saved; the amplitude of temperature or pressure change in the purification process is far less than the temperature difference or pressure difference between the cryogenic adsorption method, the normal-temperature high-pressure adsorption method and the palladium membrane method, plus a moderate flushing, depressurization and regeneration method, the service life of the adsorbent is greatly extended, and the device lifetime can be increased by more than 10 years; the embodiment solves the problems of “hydrogen brittleness” fracture of palladium membrane tubes, pulverization of adsorbents and the like existing in the prior art such as palladium membrane method and cryogenic adsorption method, and overcomes the difficult problems of high price and huge investment in palladium membranes, insulation, corrosion resisting materials and the like for ultralow temperature adsorption equipment, complex manufacturing process for large ultralow temperature equipment and palladium membranes (tubes), short device lifetime and the like; since it is IncPSA, the cyclic operations of adsorption and regeneration are easier to realize; raw gas of pure hydrogen up to tens of thousands of standard volume per hour can be purified through adsorption by one tower or simultaneous adsorption by a plurality of towers with regeneration by other towers, so that the production output of ultra-pure hydrogen is expanded, and the embodiment solves the technical and economic problem of smaller production scale due to high equipment investment, huge maintenance cost, complicated manufacturing process, smaller equipment unit volume or flux of specific surface area of palladium membrane, as well as shorter service life of adsorbents and palladium membranes and the like in the existing cryogenic adsorption, normal-temperature high-pressure adsorption, palladium membrane method and other processes; this embodiment has low requirements for raw hydrogen and high adaptability, thereby expanding various sources of raw materials of hydrogen, including hydrogen with methanol, natural gas, coal, biogas and various kinds of hydrogen-containing industrial tail gas as raw materials with production content ≥98-99%, and the technical problem of too high requirements for the quality of raw hydrogen in the prior art is solved; there is no loss in the pressure of raw hydrogen and ultra-pure product hydrogen, the ultra-pure hydrogen with pressure in the intercooling temperature range is more suitable for use in electronic semiconductor processing, and the embodiment solves the problem existing in the palladium membrane method that the pressure of raw pure hydrogen is high but the pressure of the ultra-pure product hydrogen is almost zero, the back end needs to be pressurized before use, so the energy consumption is high; meanwhile, the embodiment also overcomes the technical problem existing in the cryogenic adsorption and normal-temperature high-pressure adsorption methods that the energy consumption is further increased due to the fact that the temperature or pressure of the raw pure hydrogen differs considerably from the temperature or pressure required for ultra-pure product hydrogen, which requires subsequent heating or depressurization; the operating temperature is −80-−10° C., the operating pressure is 0.05-3.5 MPa, this intercooling temperature range can be achieved by using ordinary refrigerators, the corresponding equipment materials such as adsorption, pipelines and valves use general carbon steel or conventional stainless steel and the like applicable to the intercooling range, and the embodiment solves the technical and economic problems of ultra-low temperature resistance, high pressure resistance, low temperature corrosion resistance and the like required for equipment materials in the cryogenic adsorption, normal-temperature high-pressure adsorption and palladium membrane methods.

### Embodiment 5

As shown in FIG. 5, a method for gas separation, purification and clarification by FTrPSA, applied to the field of pure oxygen preparation with the air as the raw gas, comprises the following operations in turn:

(1) Pretreatment procedure: air without dust particles is obtained after dust removal of the raw gas, then the air without dust particles is fed into an air turbine compressor, compressed to 0.1-0.6 MPa, supplied into an air cooling tower for cleaning and precooled to −60-−10° C.

(2) Shallow-cooling low-pressure adsorption concentration procedure: after the pretreatment procedure, the raw gas is fed into the shallow cooling low-pressure adsorption system for adsorption, 6 adsorption towers are used for alternate cyclic operation at the operating temperature of −60-−50° C. and the operating pressure of 0.1-0.6 MPa, and ensure continuous entry of raw gas after cleaning and precooling. From the non-adsorbed phase in this procedure, the intermediate gas with oxygen content of 60-80% and the N2-rich concentrated gas formed by desorption and regeneration by flushing or depressurization are obtained.

(3) Intermediate gas treatment procedure: the intermediate gas enters the subsequent hydrogen refining procedure, that is, entering the cryogenic refining procedure which mainly consists of impurity removal, expansion refrigeration and rectification units of the molecular sieve adsorption system. In the impurity removal unit of the molecular sieve adsorption system, the intermediate gas (oxygen-rich gas) from the shallow cooling low-pressure adsorption concentration procedure enters an alternately used molecular sieve adsorber. In the molecular sieve adsorber at the operating temperature of −60-−10° C. and the operating pressure of 0.1-0.6 MPa. the moisture, carbon dioxide (CO2), hydrocarbons (CnHm) and other impure substances and a small amount of N2 are adsorbed by the molecular sieve, so that relatively pure oxygen is obtained. The process comprises selectively adsorbing the moisture, CO2, CnHm and other impurities with the molecular sieve at a higher level and removing them by heating and regeneration; in the expansion refrigeration unit, the intermediate gas after impurity removal by the molecular sieve adsorption system is supplied into an expander, the gas expands to overcome the molecular attraction, consume molecular kinetic energy and reduce the gas temperature. After expansion and cooling via the air heat exchanger, the gas is supplied into the rectification unit to participate in rectification; the rectification tower of the rectification unit is divided into an upper tower and a lower tower, the oxygen-rich gas (intermediate gas) after expansion and refrigeration is fed into the upper tower for rectification after cooling via the air heat exchanger after expansion, high purity N2 is obtained at the top of the upper tower, O2 with purity above 99.5% is obtained at the bottom of the upper tower, then the gas comes out of a cooling box after entering a main heat exchanger for reheating via the air heat exchanger after expansion, and enters the product O2 pipe network after pressurized to 3.0 MPa(G) through the oxygen turbine compressor. Liquid air is obtained at the bottom of the lower tower, the liquid air extracted from the lower tower is fed to the upper tower after being fed into a super cooler for supercooling, and further rectified through the upper tower as reflux. Liquid nitrogen is obtained at the top of the lower tower, and a part of the liquid nitrogen is extracted as the regeneration gas in the process of impurity removal and regeneration through the molecular sieve after heat exchange. Alternatively, a part of liquid nitrogen is extracted depending on the operation situation of the upper tower and used directly as reflux after heat exchange to flow in via the upper half of the upper tower for further rectification. The yield of pure oxygen preparation from air is more than 92-96% by the FTrPSA gas separation, purification and clarification methods.

(4) Concentrated gas treatment procedure: the concentrated gas is fed into the subsequent N2 recycling procedure.

This embodiment solves the problems of low yield of product O2, further prominent contradiction between normal-temperature PSA and regeneration, incomplete regeneration and shortened service life of adsorbents in the conventional PSA-based method; the FTrPSA method solves the problems of high energy consumption, high equipment investment and maintenance costs, potential safety hazard of local aggregation of CnHm impurities and the like in the cryogenic rectification method; the processes of molecular sieve adsorption, impurity removal and clarification in the shallow-cooling low-pressure adsorption concentration procedure and the cryogenic rectification procedure proceed in the shallow-cooling low-pressure range, so that the normal-temperature feeding modes by the conventional PSA-based method, cryogenic rectification method and a simple combination of both methods are greatly changed, the self-contained energy of raw gas is utilized in a gradient manner, and the cooling load of the subsequent cryogenic rectification system is greatly reduced; through the shallow-cooling low-pressure adsorption concentration procedure, first, O2 in the air is concentrated to 60-80%, then supplied into the cryogenic rectification system, so that the air volume is reduced by more than ⅔, the load of the subsequent cryogenic rectification procedure is further reduced, the problems of high investment and high energy consumption in the cryogenic rectification method are solved, and the serious contradiction between purity and yield existing in PSA extraction of O2 at normal temperature is solved; on the premise of high yield of O2, the working load of adsorbents is reduced, the service life of adsorbents is greatly prolonged, the problem of incomplete regeneration existing in the PSA process at normal temperature is solved, in particular, the impurities difficult to desorb, such as trace hydrocarbons (CnHm), carbon dioxide (CO2), carbon monoxide (CO) and the like existing in the air are thoroughly desorbed, which avoids the impurities brought into the back-end cryogenic rectification procedure, causing the potential safety hazards as a result of CO2 easy to freeze at low temperature and block the pipelines and those caused by CnHm easy to locally aggregate; compared with the simple combination of normal-temperature PSA and cryogenic rectification methods, there is still a conventional molecular sieve dewatering and impurity removal clarification procedure provided as the second protection device for cryogenic rectification in the FTrPSA method before entry into the cryogenic rectification section, which further avoids the big problem that the impurities such as CnHm and CO2 easy to cause potential safety hazards directly enter the cryogenic rectification procedure. The regeneration pressure in the front-end concentration procedure is also relieved, so that the adsorption and regeneration cycles of the front-end adsorption concentration procedure are much more easily matched and balanced.

In the FTrPSA process, the oxygen-rich gas cooled and liquefied enters the upper tower of cryogenic rectification rather than entering the lower tower in the same way as in the simple combination of PSA and cryogenic rectification in order to avoid low efficiency of the lower tower due to seriously uneven distribution of material flows at the lower tower caused by the oxygen-rich gas entering the lower tower with the concentration of 60-80%, as well as relatively surplus separating power of the upper rectification tower, resulting load mismatching of the whole tower, unstable operation and other negative effects, and the unfavorable situation of resulting energy consumption surging rather than falling; in order to stabilize the operations of the upper and lower towers for cryogenic rectification, in addition to maintaining the original cryogenic rectification reflux, it is also possible to balance the operation and running of the upper and lower towers by setting and adjusting part of the oxygen-rich gas into the upper half of the lower tower depending on the operation situations to achieve the smooth operation of the whole rectification tower and further reduce the energy consumption. In addition, in the method, the conventional practice is using the lower tower of cryogenic rectification as a sieve tray, a filler tower is used as in the case of the upper tower without further increasing the diameter or height of the lower tower, the tray resistance and temperature difference in the cryogenic rectification tower can also be effectively reduced, thus achieving the effects of energy saving and consumption reduction at a microcosmic level; compared with the simple combination of normal-temperature PSA and cryogenic rectification, this embodiment allows extraction of a part of liquid nitrogen from cryogenic rectification as the regenerated gas for impurity removal and clarification with the molecular sieve through heat exchange, and the regeneration temperature is 25-100° C. far below the temperature of 100-300° C. required for the regeneration of impurity removal and clarification units of the molecular sieve in the conventional cryogenic rectification procedure. This embodiment can realize obvious energy saving and consumption reduction only by forming a complete set of organized whole through all units; this embodiment solves the problems of low purity and low yield of product in the conventional PSA oxygen production method, the oxygen product with purity of more than 99.5% is obtained with the yield of more than 92-96%, while the purity in the conventional PSA oxygen production method can reach 95% at most, and the oxygen yield is only 30-68%.

### Embodiment 6

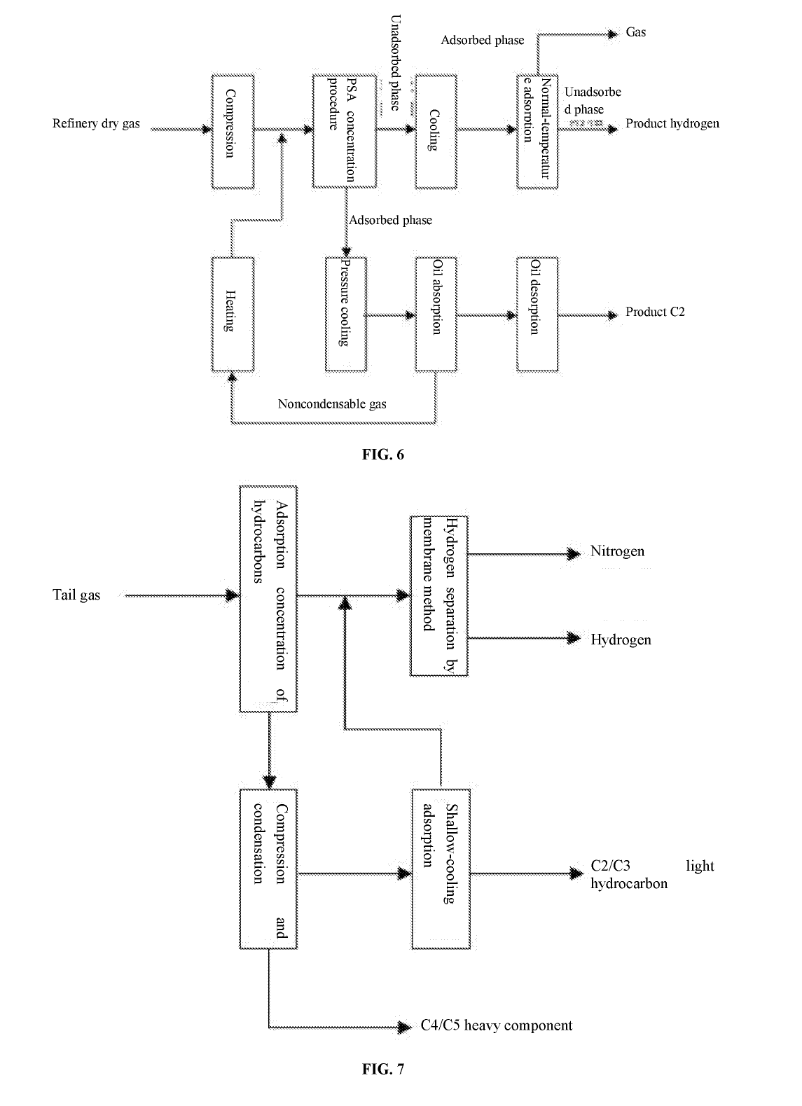

As shown in FIG. 6, a method for gas separation, purification and clarification by FTrPSA, applied to the recycling of C2 and above from refinery dry gas, comprises the following operations in turn:

(1) PSA concentration procedure: the refinery dry gas with the temperature of 50-150° C., pressure of 0.5-4.0 MPa, H2 content of 35%, content of C2 and above of 16%, methane content of 38% and the content of other components of 11% is directly supplied into an adsorption concentration system via an air intake pipeline without cooling or pressurization for adsorption by the process of 6-tower connection in parallel at a operating temperature of 50-150° C. and operating pressure of 0.5-4.0 MPa, the 6 adsorption towers are alternately and circularly operated to ensure continuous entry of raw gas, one or several adsorption towers are in the adsorption state, and the remaining adsorption tower is in a regeneration state; in the adsorption tower, the exhaust from the tower top through the bed is an intermediate gas consisting of H2, methane, nitrogen and the like, the hydrocarbon components, i.e. C2 and above, are adsorbed, and the hydrocarbon-rich concentrated gas is obtained after desorption.

(2) Intermediate gas treatment procedure: the normal-temperature adsorption procedure proceeds after the intermediate gas is cooled to the normal temperature through the cooling procedure, the components such as methane, nitrogen and trace light hydrocarbons are removed, and a high-purity H2 product with volume concentration of greater than 99.99% and yield of over 93% is obtained. After desorption, a fuel gas dominated by methane is obtained.

(3) Concentrated gas treatment procedure: a concentrated gas undergoes the procedures of pressure cooling, oil absorption and oil desorption in turn: a. pressure cooling procedure, the concentrated gas is pressurized to 1.5-4.0 Mpa by a compressor and then cooled to 10-40° C. to achieve the required feeding conditions for oil absorption, wherein the volume of concentrated gas obtained from the adsorption concentration procedure of C2 and above is much less than that of raw gas, so that the load of pressure cooling in this procedure is greatly reduced, and energy consumption and equipment investment are reduced; b. oil absorption procedure, the concentrated gas pressurized and cooled through the pressure cooling procedure is fed into the oil absorption unit, the absorption liquid used for oil absorption is C3-C6 alkane, the absorption pressure is 1.5-4.0 Mpa, the absorption temperature is 10-40° C., the components C2 and above in the hydrocarbon-rich desorbed gas are absorbed by oil, and a small amount of non-condensable gas not absorbed by oil is discharged from the top of the absorption tower; the non-condensable gas is a mixture containing methane, hydrogen, C2 and the like; the non-condensable gas flows back to the PSA concentration procedure after being heated to 60-80° C.; if refinery dry gas is saturated dry gas, that is, without or with light alkene components, i.e. trace C2 and above, such as ethylene, propylene and other alkenes, with only light alkanes, i.e. C2 and above, such as ethane and propane, propane can be used as absorption liquid, so that the subsequent oil desorption procedure can be omitted. In the case of unsaturated dry gas, C4 (butane) or gasoline is generally used as an absorbent. In addition, the small amount of non-condensable gas discharged from the top of the absorption tower is heated to 50-150° C. by heat exchange with the heated intermediate gas discharged from the top of the adsorption tower in the concentration procedure of C2 and above, and mixed with the raw gas to enter the PSA concentration procedure; c. Oil desorption procedure, the absorption liquid produced from the oil absorption procedure is fed into the desorption tower for desorption by means of heating and depressurization; after desorption, the absorption liquid is circulated to the absorption tower for reuse and optimized by the regulation of pressure and temperature, and the C2 product obtained has the concentration of more than 98% and yield of more than 92-96%.

In this embodiment, the refinery dry gas is firstly separated into concentrated gas and intermediate gas through the adsorption and concentration procedure of C2 and above in the FTrPSA process, the self-contained energy of the refinery dry gas is fully utilized, then the concentrated gas and the intermediate gas are separated respectively, wherein the concentrated gas undergoes the subsequent recycling procedure consisting of pressure cooling, oil absorption and oil desorption units in turn, so that C2 and above products with a concentration of more than 98% are obtained; the non-condensable gas produced in the oil absorption procedure is discharged from the top of the absorption tower, the non-condensable gas is heated to 50-150° C. before flowing back to the concentration procedure of C2 and above and recycled, and the recycling rate is improved, up to above 92-96%; after the intermediate gas undergoes the subsequent H2 refining (fine removal of C2 and above) procedure consisting of cooling and normal-temperature adsorption units, a high purity H2 product with a concentration of more than 99.99% is obtained, with yield up to over 92%; the methane based fuel gas is obtained after desorption and directly fed to the combustion pipe network for full utilization of resources; the present invention adopts the FTrPSA process, which makes the cyclic operations of adsorption and desorption easier compared with the conventional PSA, with better desorption effects and increased service life of adsorbents; meanwhile, a lot of H2, methane and other inert components relative to C2 and above are separated by the adsorption concentration procedure of C2 and above, so that the amount of hydrocarbon-rich gas is reduced, the energy consumption of subsequent compression condensation is reduced, and the volume of the equipment for separating hydrocarbon substances of C2 and above is also reduced; Therefore, this embodiment solves the problems of requirement for complex pretreatment device, long flow, high energy consumption, more limitations on effective components and temperature and pressure ranges of raw gas, low product recycling rate and purity, high investment and unstable operation in the existing processes such as recycling of H2, C2 and above from the current refinery dry gas existing in oil absorption, PSA, cryogenic separation, membrane separation, mutual combination and other methods.

### Embodiment 7

As shown in FIG. 7, a method for gas separation, purification and clarification by FTrPSA, applied to the recycling of ethylene and propylene from polyolefin tail gas, comprises the following main procedures:

(1) PSA concentration procedure: the raw gas is supplied into a PSA system consisting of 3-10 adsorption towers for adsorption, the operating temperature is maintained at 40-150° C., the operating pressure during adsorption is 0.03-3.5 MPa. a plurality of adsorption towers are alternately and circularly operated to ensure continuous entry of the raw gas, one or several adsorption towers are in the adsorption state, and the remaining adsorption tower is in a regeneration state; in the adsorption tower, the exhaust from the tower top through the bed is an intermediate gas consisting of N2 and a small amount of H2, the hydrocarbon components are adsorbed, and the hydrocarbon-rich concentrated gas is obtained after desorption and regeneration;

(2) Intermediate gas treatment procedure: The intermediate gas is pressurized to 0.5-2.0 MPa before entering the hydrogen membrane separation unit, H2 permeates the membrane layer and is separated through the membrane, a hydrogen-rich gas is obtained from the hydrogen separation membrane permeation side, and a N2 product is obtained from the non-permeation side;

(3) Concentrated gas treatment procedure: a. the concentrated gas is fed into the compression condensing unit, its pressure is raised to 0.5-2.5 MPa, the temperature is reduced to −10-20° C., a liquid C4/C5 heavy hydrocarbon component and a non-condensable gas are obtained, wherein the non-condensable gas enters the next unit for subsequent alkene recycling procedure—shallow-cooling PSA procedure; b. the non-condensable gases obtained from the compression condensing procedure mainly contain C2/C3 hydrocarbon substances and a small amount of N2 and H2; after heating to 5-20° C., the gases are fed into the shallow-cooling PSA system, the temperature in the adsorption tower for shallow-cooling PSA is in the shallow-cooling range 5-20° C., the adsorbed phase gas obtained is mainly C2/C3 light hydrocarbon after shallow-cooling PSA and desorption, and the non-adsorbed phase gas is supplied into the hydrogen membrane separation unit for recycling.

Heated and pressurized polyolefin tail gas directly enters the FTrPSA system in this embodiment without cooling condensation, pressure raising and reducing equipment and other pretreatment procedures, so that the energy consumption is greatly reduced, the pretreatment procedure investment is saved, the self-contained energy of raw gas is fully utilized, and the embodiment is suitable for low pressure separation of polyolefin tail gas where no breakthrough can be made on the balance having achieved between hydrocarbon components such as N2, C2 and above by using the conventional pressurization condensing pretreatment so that the separation target is difficult to complete in the subsequent separation procedure; the vast majority of N2 is separated through the adsorption concentration procedure of hydrocarbons, the hydrocarbon substances such as C2 and above in the tail gas are concentrated, reducing the amount of hydrocarbon-rich concentrated gas, so that the concentrated gas of hydrocarbons such as C2 and above meet the requirements for subsequent compression, condensation and feeding; the embodiment overcomes the advantage that it is difficult for general compression condensation to completely separate C4/C5 from concentrated gas of hydrocarbons such as C2 and above through liquefaction, so that the energy consumption of subsequent compression condensation is reduced, and the volume of the device for separation of hydrocarbon substances is also reduced; the coupling of compression condensation and shallow-cooling PSA not only improves the recycling rate of hydrocarbon substances, but also segments the hydrocarbon substances into two parts: C4/C5 heavy component and C2/C3 light component, and the energy consumption is reduced; the intermediate gas consisting of a large amount of N2 and a small amount of H2 and flowing out from the adsorption tower in the adsorption concentration procedure of hydrocarbons can directly enter without pressurization under the condition of operating pressure greater than or equal to 0.5 MPa while recycling high purity nitrogen to realize multi-component recycling of tail gas; this embodiment uses the FTrPSA process, so that the service life of adsorbents is increased, the service life of adsorbents is more than 10 years, with high degree of automation, stable operation and low investment.

### Embodiment 8

As shown in FIG. 8, a method for gas separation, purification and clarification by FTrPSA, applied to the recycling of C2 and above from the tail gas of low temperature methanol washing, comprises the following main steps:

(1) Pressure Swing Adsorption Concentration Procedure

a. At normal pressure, the composition of the tail gas of low temperature methanol washing is content of non-methane hydrocarbon substances 0.6%, N2 10.1%, C02 88%, and the rest 1.3%; the feeding temperature is 40-150° C., and the feed flow rate is 100,000-1,200,000 m3/hour;

b. After the pressure adjustment through the fan, the tail gas of low temperature methanol washing is fed into the PSA system consisting of 8 adsorption towers connected in series, from the bottom of the adsorption tower and then adsorbed at 30-100 KPa and operating temperature of 40-150° C., one or several adsorption towers are in the adsorption state, and the remaining adsorption tower is in a regeneration state;

c. The unadsorbed CO2 and part of N2 are discharged from the top of the adsorption tower to meet the emission limit 120 mg/m3 specified by the national standard and become acceptable effluent gas, emptied on the spot and discharged into the atmosphere;

d. The components in the adsorbed phase are the hydrocarbon substances concentrated to 10-30% and 70-90% CO2, the hydrocarbon-rich gas is obtained by emptying and desorption through a vacuum pump, and then the gas enters the subsequent concentrated gas treatment procedure;

(2) Concentrated Gas Treatment Procedure:

a. Primary cooling unit, the temperature of concentrated gas from the PSA concentration procedure is 40-150° C., and the hydrocarbon-rich desorbed gas is reduced to 20-25° C. by means of circulating water cooling;

b. Secondary cooling unit, the temperature of hydrocarbon-rich desorbed gas is further reduced from 20-25° C. to −5-5° C. with chilled water to remove most of C4 and above and part of C2 and C3 components;

c. Cold alcohol absorption unit, the cooled hydrocarbo-rich desorbed gas enters the cold alcohol absorption unit, the absorbent used for cold alcohol absorption is methanol, the absorption pressure is 3.0-3.5 MPa, and the absorption temperature is −5-5° C.; CO2 in the hydrocarbon-rich desorbed gas is absorbed by methanol, unabsorbed hydrocarbon substances are discharged from the top of the absorption tower, and returned to the secondary cooling unit to obtain the hydrocarbon components.

d. Cold alcohol desorption unit, the desorption pressure is normal pressure, and the temperature is −5-5° C. The desorbed alcohol absorbent is pressurized to 3.0-3.5 MPa and returned to the cold alcohol absorption unit for recycling. The desorbed CO2 is recycled by flashing.

In this embodiment, the low temperature methanol tail gas is fed into the multi-tower PSA device for adsorption concentration of hydrocarbons. Since the operating temperature of 40-150° C. is used, the low temperature methanol tail gas is directly fed without cooling, this not only reduces energy consumption, but also enables hydrocarbon substances (0.6%) with a very low content in the tail gas to be adsorbed effectively as adsorbates and concentrated to 10-30%; meanwhile, the embodiment avoids the technical bottlenecks in the conventional TSA that it is difficult to select an inert heat carrier as a heat source, adsorption and regeneration temperatures need to periodically alternate, and the consumption of a lot of heat is required. This procedure uses the PSA concentration procedure for its characteristics of enhanced adsorption capacity, complete regeneration and no periodic alternation in temperature, so that the service life of adsorbents is increased. The embodiment solves the problem that the content of hydrocarbon components in the adsorbed phase is low, so it is not easy to adsorb or difficult to desorb. A hydrocarbon-rich concentrated gas rich in hydrocarbon substances is obtained. The main purpose of using the PSA concentration procedure is to concentrate hydrocarbon substances, and discharge most of CO2 gas that meets the limit of hydrocarbon content specified by the national standard. This part of effluent gas also allows CO2 recycling by flashing. The temperature of the concentrated gas from the PSA concentration procedure is 40-150° C., and the gas needs to be cooled. In this embodiment, the temperature is reduced to the normal temperature 20-25° C. by means of circulating water cooling, and the circulating water is easy to find with low cost. The intermediate gas is cooled by circulating water, which is favorable for control of production cost; its temperature is reduced from 20-25° C. to −5-5° C. with chilled water to remove most of C4 and above and part of C2 and C3 components. In this process, most of C4 and above and part of C2 and C3 components cooled to −5-5° C. become liquid and are separated from the mixed gas, depending on the boiling points of different alkane components. The cooled hydrocarbon containing gas enters the cold alcohol absorption unit, the absorbent used for cold alcohol absorption is readily-accessible methanol, the absorption pressure is 3.0-3.5 MPa, and the absorption temperature is −5-5° C. According to the principle of “like dissolves like”, CO2 in the mixed gas is absorbed by methanol, unabsorbed hydrocarbons are discharged from the top of the absorption tower and returned to the secondary cooling procedure to recycle the hydrocarbons. Meanwhile, the absorption liquid which absorbs CO2 is fed into the cold alcohol desorption unit for normal pressure desorption, the resulting methanol is pressurized and returned to the cold alcohol absorption unit for recycling, and the desorbed CO2 can be recycled by flashing.

This embodiment solves the technical bottlenecks that adsorption is difficult due to the low content and low partial pressure of low concentration hydrocarbons in the low temperature methanol washing tail gas at normal temperature and pressure existing in the conventional PSA-based method, and that it is difficult to match up with the adsorption time and then difficult to form the cyclic operation of adsorption and regeneration desorption because it is difficult to select an inert heat carrier which does not react with hydrocarbons and CO2 component in the tail gas of low temperature methanol washing in the process of desorption, or due to large treatment capacity and too long heating-up time existing in the conventional TSA method. The embodiment is a major technological breakthrough which realizes the recycling of hydrocarbons from the tail gas of low temperature methanol washing and meets the emission standards specified by the state; with the PSA concentration procedure, the embodiment solves the problem of coadsorption of CO2 and hydrocarbon components at normal temperature and normal pressure and the technical bottleneck of low concentration hydrocarbons not easy to use PSA by using the differences between adsorption and desorption mechanisms on different adsorbents at a temperature of 40-150° C. and pressure of 30-100 KPa for easily adsorbed hydrocarbon components in the tail gas of low temperature methanol washing and the CO2 component not easy to adsorb within the medium & high-temperature and low pressure ranges, and overcomes the difficulties of high energy consumption for TSA regeneration, excessive load, difficulty in selection of inert heat carrier and the like; hydrocarbon substances are adsorbed, concentrated and recycled, so that their contents in the tail gas of low temperature methanol washing is less than the emission limit 120 mg/m3 specified in the Integrated Emission Standard of Air Pollutants; the embodiment ensures emissions up to standard, and solves the problem that the tail gas of low temperature methanol washing can neither be combusted nor recycled, and even cannot be discharged directly; a plurality of adsorption towers are provided in the PSA concentration procedure, when a part of the adsorption towers is in the adsorption state, the other part of the adsorption towers is in the regeneration state, so the continuous production of the whole hydrocarbon recycling process is realized; either a section of PSA or a section of PSA coupled with a section of TSA in series can be provided in the PSA concentration procedure, wherein the section of PSA can be provided with a plurality of adsorption towers, when a part of the adsorption towers is in the adsorption state, the other part of the adsorption towers is in the regeneration state; the first section of TSA is provided with two towers, one for adsorption, one for regeneration, so that the operating load of the first section of TSA is to further relieved, and it is especially suitable for conditions with treatment capacity of more than 1 million m3/hour; for device regeneration in the PSA concentration procedure, a vacuum pumping mode is used at medium temperature for desorption without any heat source or regenerated gas; compared to the conventional TSA in need of a high temperature inert heat carrier as the heat source, the method saves a lot of energy and realizes the function of energy saving; the used alcohols, such as methanol, ethanol and the like and a mixture of lower alcohols, are used as absorbents; since methanol, ethanol and mixture of lower alcohols have small molecular weights, the absorbing capacity for CO2 in concentrated hydrocarbon-rich concentrated gas and the selectivity are good at a temperature of −20-20° C. and pressure of 1.0-4.0 Mpa, and in this way, CO2 is conveniently separated from hydrocarbons, the unabsorbed hydrocarbons are returned to the recycling procedure and recycled, and then the method overcomes the problem of coabsorption when CO2 is absorbed while hydrocarbons are absorbed with the conventional cold oil absorbents. Also, alcohol absorbents are easily accessible, so that the hydrocarbon recycling rate in the hydrocarbon recycling procedure is up to 90-95%; meanwhile, the regeneration and recycling of alcohol absorbents are facilitated through the cold alcohol desorption procedure, wherein desorbed CO2 is recycled by flashing to realize energy saving and emission reduction.

### Embodiment 9

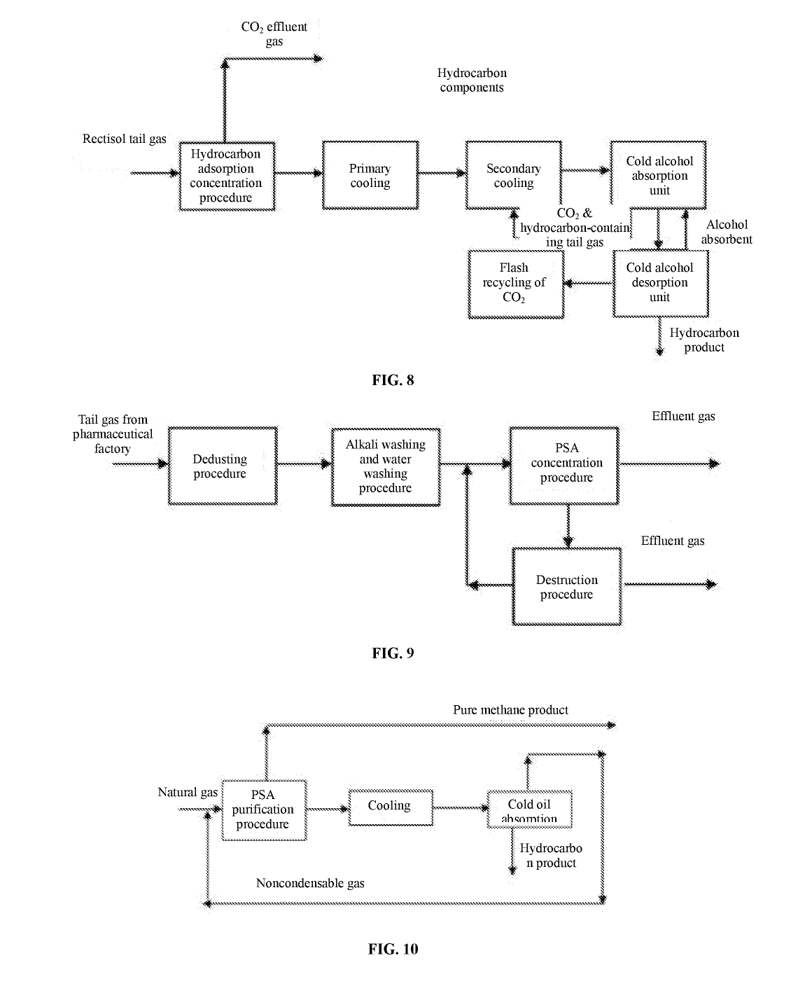

As shown in FIG. 9, a method for gas separation, purification and clarification by FTrPSA, applied in the field of medical tail gas clarification VOCs, comprises the following main procedures:

(1) Pretreatment procedure, comprising two procedures, i.e. dust removal procedure and cooling gas washing procedure: a. dust removal procedure: tail gas from a spray drying section of pharmaceutical factory enters a dust collector, dust with large particles and high specific gravity in the tail gas is settled down due to the effect of gravity, falling into an ash bucket, when gas containing fine dust passes the filter material, dust is intercepted, and dust-free tail gas from pharmaceutical factory is obtained after the gas passes through the dust collector; b. cooling and gas washing procedure: the dust-free tail gas from pharmaceutical factory is fed into a tower washing system, acid gases such as SO2 and H2S in the dust-free tail gas from pharmaceutical factory are removed, and the temperature of the high-temperature dust-free tail gas from pharmaceutical factory is reduced from 300° C.-500° C. to 60-150° C.

(2) Medium-temperature PSA concentration procedure: In the medium-temperature PSA concentration procedure, the dust-free tail gas from pharmaceutical factory after the cooling and gas washing procedure is fed from the bottom of the adsorption tower followed by the medium-temperature PSA concentration, at a adsorption pressure of 30-80 KPa and operating temperature of 60-150° C., and 8 adsorption towers are alternately and circularly operated to ensure continuous entry of the dust-free tail gas from pharmaceutical factory; unadsorbed air and CO2 are discharged from the top of the adsorption tower. The content of VOCs is detected. The gases are discharged into the atmosphere on the spot if the total VOCs meet the specified emission standard, i.e., below 6 ppm. The VOCs in the adsorbed phase are condensed to 10-30%, concentrated VOCs-rich gas is obtained by vacuum pump evacuation, desorption and regeneration, followed by the concentrated gas treatment procedure.

(3) Concentrated gas treatment procedure: the concentrated VOCs-rich gas obtained by desorption and regeneration in the medium-temperature PSA concentration procedure is delivered to combustion, the combusted tail gas is tested, the tail gas up to testing standard is directly discharged into the atmosphere, and the unacceptable tail gas is returned to the medium-temperature PSA concentration procedure to continue with the recycling treatment.

According to this embodiment, the self-contained energy of the raw gas is effectively utilized in a gradient manner by the FTrPSA method, without wasting the energy of heating required for regeneration by reducing the temperature of the raw gas from a high temperature to the low temperature range of adsorption and clarification by the conventional TSA method; this embodiment solves the contradiction between easy adsorption and difficult desorption existing in the conventional PSA technology and the environmental health problems brought about by other existing technologies which cannot effectively remove VOCs and their bitter, astringent and pungent odor; the energy consumption and cost for device operation are low, and the service life of adsorbent is long: 1) This embodiment greatly reduces the investment and energy consumption of the device, and the device lifetime is over 10 years; since the principle is PSA and desorption proceeds in a depressurization mode without a lot of regeneration heat carrier gas and heat source, the energy consumption and cost of device operation are greatly reduced; this embodiment can realize complete desorption at a operating temperature of 60-150° C. to avoid residual VOCs poisoning the adsorption bed; there is no periodic temperature variation between heating and cooling, so that the long adsorbent life is guaranteed; 2) clarification of the raw gas is started without reducing to a very low temperature; the tail gas temperature is about 300° C. after spray drying, and its temperature is reduced to 60-150° C. by washing or alkali washing after bag dust-cleaning; the temperature needs to be reduced to below the normal temperature 40° C. as compared with the conventional process, and the present invention reduces the sensible heat waste of raw gas and the circulation of circulating solution; zero emission of tail gas is achieved: the concentrated gas in the FTrPSA method enters the subsequent clarification procedure—destruction procedure for combustion, the combusted tail gas is tested, the tested tail gas up to the standard is directly discharged into the atmosphere, the tail gas not up to the standard is returned to the medium-temperature PSA concentration procedure to continue with recycling treatment, so that the FTrPSA procedure forms a complete closed system, VOCs and other impurities in the tail gas are completely destroyed, so that zero emission of tail gas is achieved; the process flow is short and simple without strict pretreatment, the adaptability to raw materials is high, and the method solves the problems of long and complex flow, requirement for strict pretreatment and the like in the prior art.

### Embodiment 10

As shown in FIG. 10, a method for gas separation, purification and clarification by FTrPSA, applied in the field of methane preparation from natural gas—natural gas clarification, comprises the following main steps:

(1) Medium-temperature PSA concentration procedure: This procedure consists of a plurality of adsorption towers connected in series or in parallel or connected in series and in parallel, wherein some of the adsorption towers are in an adsorption state, and the remaining adsorption tower is in a regeneration state; the filler in the adsorption towers is one or several kinds of activated carbon, silica gel, activated aluminium oxide and molecular sieve; natural gas is used as the raw gas, fed from the bottom of the adsorption towers and adsorbed at a adsorption pressure of 0.05-3.5 MPa and operating temperature of 60-150° C., and the alternate cyclic operation of a plurality of adsorption towers guarantees continuous entry of natural gas; unadsorbed methane (CH4) is discharged from the top of the adsorption tower, and the resulting pure methane product is stored in a methane product tank; hydrocarbon components such as C2 and above in the adsorbed phase are concentrated to 15-30%, the hydrocarbon-rich gas is obtained by vacuum pump evacuation and desorption, followed by the concentrated gas treatment procedure.

(2) Concentrated gas treatment procedure, after acid components are removed, the directly pressurized hydrocarbon-rich concentrated gas is fed into the fuel pipe network for combustion and use as a fuel gas, or the hydrocarbon-rich concentrated gas is recycled, and the concentrated gas recycling procedure includes three sections, i.e. primary cooling, secondary cooling and cold oil absorption:

a. Primary cooling: The temperature of the hydrocarbon-rich concentrated gas from the medium-temperature PSA concentration procedure is 60-150° C., and the hydrocarbon-rich concentrated gas is cooled to the normal temperature;

b. Secondary cooling: The temperature of the concentrated gas is further reduced to −10-10° C., and most C4 and above and part of C2 and C3 components are removed;

c. Cold oil absorption: The cooled hydrocarbon-rich concentrated gas is fed into the cold oil absorption tower, and the frozen C3-C6 stable light oil is used as the absorbent with absorption pressure of 1.5-4.0 MPa and absorption temperature of −10-10° C.; C2 and above in the hydrocarbon-rich concentrated gas are absorbed, non-condensable gas is formed by a small amount of unabsorbed methane and the like, discharged from the top of the cold oil absorption tower, and returned to the medium-temperature PSA concentration procedure after heat exchange to continue with cyclic treatment and recycling of methane; the product components of C2 and above are obtained through desorption regeneration of absorption liquid, while the absorbent can be recycled.

According to this embodiment, adsorption and desorption proceed at 60-150° C. by using the medium-temperature PSA concentration procedure. The embodiment solves the problem that the adsorbed phases C2 and above cannot be desorbed or difficult to desorbed, and avoids causing inactivation of adsorbents in the tower due to long-time accumulation of C2 and above in the adsorption tower; since the temperature is 60-150° C. in both of adsorption and desorption processes, the temperature does not alternate periodically, so that the service life of the adsorbent is over 10 years; desorption regeneration proceeds at medium temperature by way of vacuuming without heat source or regenerated gas; compared with conventional TSA requiring high temperature steam or hot regenerated gas as the heat source, the use of the medium-temperature PSA concentration procedure saves a lot of heat energy and regenerated gas, and achieves purpose of energy saving.

The embodiment makes full use of the self-contained energy of natural gas, uses the medium-temperature PSA concentration procedure, and solves the problem that adsorbed phase C2 and above cannot be desorbed or difficult to desorb in adsorption and clarification of natural gas, the service life of the adsorbent in the medium-temperature PSA concentration procedure is greatly increased to more than 10 years: for the conventional PSA, C2 and above are not easy to desorb and long-term accumulation of C2 and above causes inactivation of the adsorbent in the tower, so the adsorbent lifetime is short (less than 2 years); the periodic temperature alternation of the conventional TSA is also the reason for short service life of the adsorbent (less than 3 years); a plurality of adsorption towers are arranged in the medium-temperature PSA concentration procedure, when a part of the adsorption towers is in the adsorption state, the other part of the adsorption towers is in the regeneration state, so the large-scale and continuous production of the whole hydrocarbon recycling process is realized, the purity of product methane is over 99%, and the yield is more than 90%; the regeneration step in the medium-temperature PSA concentration procedure is desorption at medium temperature by way of vacuuming without heat source or regeneration heat carrier gas; compared with the conventional TSA requiring high temperature steam or heat regenerated gas as heat source, the method saves a lot of heat energy and regenerated gas, and realizes the purpose of energy saving; the investment and running costs are low, and the method solves the problem of extremely high investment and energy consumption for preparation of pure methane by the cryogenic rectification method in the prior art.

Obviously, the above-mentioned embodiment is only part of the embodiment in the present invention rather than the whole embodiment. Based on the embodiment recorded in the present invention, with respect to all other embodiments obtained by those skilled in the art without paying creative work, or the structural changes made under the inspiration of the present invention, all technical solution that are identical or similar to the present invention fall into the protection scope of the present invention.

1: A method for gas separation, purification and clarification by FTrPSA (full temperature range-pressure swing adsorption), comprising following procedures proceed in turn:
1) executing PSA (pressure swing adsorption) concentration procedure: introducing raw gas into a PSA system; wherein the PSA system uses a multi-tower series or parallel connection process for alternate cyclic operation, with an adsorption temperature maintained at −80-200° C. and an adsorption pressure at 0.03-4.0 MPa; the raw gas is divided into two paths, intermediate gas and concentrated gas, which are separated for subsequent treatment;
2) executing intermediate gas treatment procedure: wherein the intermediate gas is an unadsorbed gas in the PSA concentration procedure; which is discharged directly or stored, or refined in the refining procedure before being discharged or stored; and
3) executing concentrated gas treatment procedure: wherein the concentrated gas is the adsorbed gas in the PSA concentration procedure; the adsorbate component is further recycled or removed in the adsorbate recycling and removal procedure.

2: The method for gas separation, purification and clarification by FTrPSA according to claim 1, wherein before the PSA concentration procedure, the raw gas enters the pretreatment procedure for dedusting, preliminary concentration or preliminary purification, and a temperature of the raw gas is adjusted to −80-200° C.

3: The method for gas separation, purification and clarification by FTrPSA according to claim 1, wherein the raw gas is one or more of following components:
1) components with a low boiling point: hydrogen, nitrogen, oxygen, carbon monoxide, methane, argon and helium;
2) hydrocarbon components with a high boiling point: ethane, ethylene, propane, propylene, C4 and above;
3) oxygen-containing compound components volatile or easy to form azeotrope when meeting water: alcohols, ethers, ketones, esters, benzene and naphthalene;
4) water, carbon dioxide, ammonia and ammonia compounds, sulfur and sulfur compounds, oil and tar, starch, macromolecular organic compounds and proteins, and
5) mixed tail gas: ethylene cracking gas, ethylene tail gas, refinery dry gas, coke oven gas, hydrogen-sulfide-containing hydrogen-rich gas, rectisol tail gas, synthetic gas, purge gas from ammonia synthesis loop, methanol tail gas, overhead gas, carbon-monoxide-containing steelmaking tail gas, acetic acid tail gas, glycosylated tail gas, ethylene or propylene tail gas, polyolefin tail gas, chlor-alkali tail gas, biogas, natural gas, biomass gas, flue gas, volatile organic vapor, pharmaceutical tail gas and industrial purified gas or industrial tail gas.

4: The method for gas separation, purification and clarification by FTrPSA according to claim 1, wherein the pretreatment procedure comprises one or more of following operations:
1) executing preliminary impurity removal operation: wherein moisture, oil mist and impurity components which affect the PSA concentration procedure are removed preliminarily from the raw gas by means of drying, adsorption, condensation or scrubbing-absorption;
2) executing temperature regulating operation: wherein a temperature of the raw gas is regulated by heat exchange equipment and condensing equipment; and
3) executing pressure regulating operation: wherein a pressure of the raw gas is regulated by pressure raising and reducing equipment.

5: The method for gas separation, purification and clarification by FTrPSA according to claim 4, wherein the preliminary impurity removal operation proceeds in a dust collector, water cooling tower, scrubber tower, condensing tower, rectifying tower, PSA tower or primary adsorption tower.

6: The method for gas separation, purification and clarification by FTrPSA according to claim 4, wherein the heat exchange equipment in the temperature regulating operation is a heat exchanger or a heater.

7: The method for gas separation, purification and clarification by FTrPSA according to claim 4, wherein the condensing equipment in the temperature regulating operation is a water cooling tower, scrubber tower or condensing tower, and the raw gas is cooled by means of scrubbing or water cooling or condensation.

8: The method for gas separation, purification and clarification by FTrPSA according to claim 4, wherein the pressure raising and reducing equipment in the pressure regulating operation is a pressure reducer, throttle valve, blower or compressor.

9: The method for gas separation, purification and clarification by FTrPSA according to claim 1, wherein the PSA concentration procedure proceeds in the PSA system which consists of N adsorption towers, and N is a natural number greater than 1; wherein 1 to N−1 adsorption towers are in an adsorption state, and the remaining adsorption tower is in a regeneration state; the adsorption towers are formed by connection of the N towers in series or in parallel or a combination thereof for alternate cyclic operation.

10: The method for gas separation, purification and clarification by FTrPSA according to claim 9, wherein an adsorption mode of the adsorption tower is a concentration-based feed adsorption or a staged adsorption or a combination thereof.

11: The method for gas separation, purification and clarification by FTrPSA according to claim 9, wherein a regeneration mode of the adsorption tower is one or more of evacuation, constant pressure, flushing and displacement gas.

12: The method for gas separation, purification and clarification by FTrPSA according to claim 11, wherein a filler in the adsorption tower is one or more of activated carbon, silica gel, activated aluminium oxide and molecular sieve.

13: The method for gas separation, purification and clarification by FTrPSA according to claim 1, wherein the refining procedure in the intermediate gas treatment procedure comprises one or more of catalytic oxidation reaction, catalytic reduction reaction, solvent absorption, membrane separation, PSA, TSA (temperature swing adsorption), rectification and cryogenic separation.

14: The method for gas separation, purification and clarification by FTrPSA according to claim 13, wherein a gas component from the refining procedure is discharged directly, stored or returned to the PSA concentration procedure for further adsorption treatment.

15: The method for gas separation, purification and clarification by FTrPSA according to claim 14, wherein the gas component from the refining procedure is regulated such that a temperature and a pressure of the gas component reaches an operating temperature and an operating pressure of the PSA concentration procedure before returning to the PSA concentration procedure.

16: The method for gas separation, purification and clarification by FTrPSA according to claim 1, wherein the adsorbate recycling and removal procedure comprises one or more of catalytic oxidation reaction, catalytic reduction reaction, solvent absorption, membrane separation, PSA, TSA, rectification and cryogenic separation.

17: The method for gas separation, purification and clarification by FTrPSA according to claim 1, wherein the concentrated gas is pressurized by a compressor or heated or cooled by a heat exchanger before entering the adsorbate recycling and removal procedure.

18: The method for gas separation, purification and clarification by FTrPSA according to claim 17, wherein a gas component from the adsorbate recycling and removal procedure is discharged directly, stored or returned to the PSA concentration procedure for further adsorption treatment.

19: The method for gas separation, purification and clarification by FTrPSA according to claim 18, wherein the gas component from the adsorbate recycling and removal procedure is regulated such that a temperature and a pressure of the gas component reaches an operating temperature and an operating pressure of the PSA concentration procedure before returning to the PSA concentration procedure.
